(19) United States
(12) Patent Application Publication
(10) Pub. No.: US 2023/0134461 A1
(43) Pub. Date: May 4, 2023

(54) PATIENT-SPECIFIC SPINAL INSTRUMENTS FOR IMPLANTING IMPLANTS AND DECOMPRESSION PROCEDURES

(71) Applicant: Carlsmed, Inc., Carlsbad, CA (US)

(72) Inventors: Niall Patrick Casey, Carlsbad, CA (US); Michael J. Cordonnier, Carlsbad, CA (US)

(21) Appl. No.: 17/978,746

(22) Filed: Nov. 1, 2022

Related U.S. Application Data
(60) Provisional application No. 63/274,300, filed on Nov. 1, 2021.

(51) Int. Cl. A61B 34/10; G16H 10/60; G05B 19/4097

(52) U.S. Cl. CPC A61B 34/10; G16H 10/60; G05B 19/4097; A61B 2034/105; A61B 2034/108; A61B 2034/107; A61B 2034/104; G05B 2219/40418

(57) ABSTRACT

Systems and methods for designing and implementing patient-specific surgical procedures and/or medical devices are disclosed. In some embodiments, a method includes receiving a patient data set of a patient. The patient data set is compared to a plurality of reference patient data sets, wherein each of the plurality of reference patient data sets is associated with a corresponding reference patient. A subset of the plurality of reference patient data sets is selected based, at least partly, on similarity to the patient data set and treatment outcome of the corresponding reference patient. Based on the selected subset, at least one surgical procedure or medical device design for treating the patient is generated.

### CROSS-REFERENCE TO RELATED APPLICATION

The present application claims priority to U.S. Provisional Patent Application No. 63/274,300, filed Nov. 1, 2021, the disclosure of which is incorporated by reference herein in its entirety.

### TECHNICAL FIELD

The present disclosure is generally related to designing and implementing decompression systems, and more particularly to systems and methods for designing and implementing spinal decompression systems, implantation site preparation systems, procedures, instruments and/or medical devices.

### BACKGROUND

Numerous types of data associated with patient treatments and surgical interventions are available. To determine treatment protocols for a patient, physicians often rely on a subset of patient data available via the patient's medical record and historical outcome data. However, the amount of patient data and historical data may be limited, and the available data may not be correlated or relevant to the particular patient to be treated. Additionally, although digital data collection and processing power have improved, technologies using collected data to determine optimal treatment protocols have lagged. For example, conventional technologies in the field of orthopedics may lack the capability to draw upon large data sets to generate and optimize patient-specific treatments (e.g., surgical interventions and/or implant designs) to achieve favorable treatment outcomes.

Conventional treatments for spinal nerve compression also lack the capability to provide adequate treatment. Spinal nerve compression can be caused by narrowing of the spinal canal associated with thickening of ligaments, arthritis of the spine, and degeneration of spinal discs. Thickened ligaments located along the spine can thicken over time and press on nerve tissue (e.g., nerve tissue of the spinal cord, nerve roots, etc.). Arthritis often leads to the formation of bony features which can press on the spinal cord. A posterior portion of the degenerated disc can protrude through weakened fibrous outer covering and can press on the spinal cord and/or spinal nerve roots. Unfortunately, spinal nerve compression can cause lower back pain, hip pain, and leg pain and may also result in numbness, depending on the location of the compressed nerve tissue. Spinal implants can be implanted to relieve nerve compression and treat other conditions. Unfortunately, it may be difficult to implant devices to treat spinal nerve compression and other conditions.

### BRIEF DESCRIPTION OF THE DRAWINGS

The accompanying drawings illustrate various embodiments of systems, methods, and embodiments of various other aspects of the disclosure. Any person with ordinary skill in the art will appreciate that the illustrated element boundaries (e.g., boxes, groups of boxes, or other shapes) in the figures represent one example of the boundaries. It may be that in some examples one element may be designed as multiple elements or that multiple elements may be designed as one element. In some examples, an element shown as an internal component of one element may be implemented as an external component in another, and vice versa. Furthermore, elements may not be drawn to scale. Non-limiting and non-exhaustive descriptions are described with reference to the following drawings. The components in the figures are not necessarily to scale, emphasis instead being placed upon illustrating principles.

FIG. 1 is a side view of a decompression instrument positioned along a spine, according to an embodiment.

FIGS. 2A-2C are detailed side views of distal ends of decompression instruments positioned along a spine, according to an embodiment.

FIG. 3 is a network connection diagram illustrating a system for providing patient-specific medical care, according to an embodiment.

FIG. 4 illustrates a computing device suitable for use in connection with the system of FIG. 3, according to an embodiment.

FIG. 5 is a flow diagram illustrating a method for providing patient-specific medical care, according to an embodiment.

FIG. 6 is a schematic illustration of an operative setup including select systems and devices that can be used to provide patient-specific medical care, such as for performing the method described with respect to FIG. 5.

FIGS. 6A-6C illustrate exemplary data sets that may be used and/or generated in connection with the methods described herein, according to an embodiment. FIG. 6A illustrates a patient data set. FIG. 6B illustrates a plurality of reference patient data sets. FIG. 6C illustrates similarity scores and outcome scores for the reference patient data sets of FIG. 6B.

FIG. 7 is a flow diagram illustrating another method for providing patient-specific medical care, according to an embodiment.

FIGS. 8A and 8B illustrate an example of a virtual model of a patient's native anatomical configuration.

FIGS. 9A-9D illustrate an exemplary patient data set that may be used and/or generated in connection with the methods described herein, according to an embodiment.

FIGS. 10A-1-10B-2 illustrate an exemplary virtual model of a patient's spine in a pre-operative anatomical configuration and a corrected anatomical configuration. More specifically, FIGS. 10A-1 and 10A-2 illustrate the pre-operative anatomical configuration of the patient, FIGS. 10B-1 and 10B-2 illustrate the corrected anatomical configuration.

FIG. 11 illustrates an exemplary surgical plan for a patient-specific surgical procedure that may be used and/or generated in connection with the methods described herein, according to an embodiment.

FIG. 12 illustrates an exemplary surgical plan report detailing the surgical plan shown in FIG. 11 for surgeon review and that may be used and/or generated in connection with the methods described herein, according to an embodiment.

FIGS. 13A and 13B illustrate an exemplary patient-specific implant that can be used and/or generated in connection with the methods described herein, according to an embodiment.

FIG. 14 illustrates a segment of a patient's spine after several patient-specific implants have been implanted therein.

FIG. 15 is a flow diagram illustrating a method for providing patient-specific plans and/or decompression procedures, according to an embodiment.

FIGS. 16A-16B are detailed side views of distal ends of decompression instruments positioned along a spine, according to an embodiment.

FIG. 16C is a detailed side view of an implanted device, according to an embodiment.

FIG. 17 is a flow diagram illustrating a method for generating patient-specific designs and/or plans based on access paths, according to an embodiment.

### DETAILED DESCRIPTION

The present technology is directed to systems and methods for planning and implementing medical procedures and/or devices. For example, in many of the embodiments disclosed herein, a method of providing medical care includes comparing a patient data set of a patient to be treated with a plurality of reference patient data sets (e.g., data from previously-treated patients). The method can include selecting a subset of the reference patient data sets, e.g., based on the similarity of the reference patient data set to the patient data set and/or whether the reference patient had a favorable treatment outcome. The selected subset can be used to generate a decompression plan, surgical kit, instrument designs, and/or medical device designs that are likely to produce a favorable treatment outcome for the particular patient. In some decompression plans, a series of instruments are used to alter (e.g., crush, separate, cut, debulk, break, fracture, remove, or the like) tissue to eliminate, reduce, or limit compression of nerve tissue. The instruments can include patient-specific instruments designed to access and remove tissue, which may be located at sites that cannot be adequately accessed using standing instruments. A decompression plan can identify target tissue (also referred to as “targeted tissue”) for removal, access techniques, and visualization procedures for positioning the instruments, thereby preventing or limiting injury or damage to non-target tissues. The decompression plan can also include the implantation of one or more implants (e.g., artificial discs, intervertebral cages, interspinous spacers, fusions systems, etc.) designed based on the predicted outcome of the tissue removal. Fusion systems can be multilevel systems including one or more rods, screws (e.g., bone screws, pedicle screws, etc.), or the like. This comprehensive planning process can optimize the outcomes by removing tissue, repositioning anatomical elements, etc. to achieve one or more target outcomes, such as pain reduction associated with nerve compression, spine realignment, and other outcomes disclosed herein. In some embodiments, systems can correlate between patient pain, patient pathology, surgical procedures, instrument designs, device designs, and/or treatment outcomes, and these correlations are used to determine a personalized decompression protocol with a higher likelihood of success. Patient-specific instruments can also be used to prepare implantation sites by, for example, removing bone, roughening bone (e.g., roughening vertebral endplates), enlarging spaces (e.g., workspaces, intervertebral spaces, or implantation spaces), removing tissue (e.g., removing bone, soft tissue, cartilage, etc.), etc. The instruments can be configured to access abnormal working spaces associated with, for example, severely deformed spines, highly curved spines, etc. For example, tissue can be removed to create an access path for implanting implants. Instrument(s) can be designed to prepare an implantation site based on virtual models. In some embodiments, patient-specific instruments can be configured to perform a discectomy, laminoplasty, laminectomy, laminotomy, corpectomy, decompression, or combinations thereof. For example, a surgical kit can contain instruments designed to perform multiple of the foregoing procedures.

A virtual model representing the patient's anatomy can be used to design items (e.g., instrument(s)/implant(s)) to, for example, fit in a working space within the patient, manipulate tissue, access target sites, etc. Simulations can be performed to receive user input, such as hand movement, user recommendations, etc. In some procedures, a user can manually move simulation inputs, robotic hand devices, and/or hand controls (e.g., joysticks, touch pads, etc.) to simulate instrument manipulation. The system can determine manipulation parameters based on the user input and can design patient-specific items based on those manipulation parameters. The user input can include one or more of, for example, a range of motion of one or both hands of the user, a user's strength, manual instrument positioning accuracy, or the like.

In the context of orthopedic surgery, systems with improved computing capabilities (e.g., predictive analytics, machine learning, neural networks, artificial intelligence (AI)) can use large data sets to identify nerve tissue associated with pain or discomfort, define improved or optimal surgical interventions, predicted pain/discomfort reduction, and/or instrument/implant designs for a specific patient. The patient's entire data can be characterized and compared to aggregated data from groups of prior patients (e.g., questionnaire results, parameters, metrics, pathologies, treatments, outcomes). In some embodiments, the systems described herein use this aggregated data to formulate potential treatment solutions (e.g., surgical plans and/or instrument/implant designs for spine and orthopedic procedures) and analyze the associated likelihood of success. These systems can further compare potential treatment solutions to determine an optimal patient-specific solution that is expected to maximize the likelihood for a successful outcome.

In some embodiments, patient-specific kits can be designed to perform decompression procedures designed for a patient's anatomy. The present technology can identify one or more paths to access sites that contribute to compression of nerve tissue. In some embodiments, the present technology can analyze patient images to identify candidate nerve compression locations that can, for example, cause discomfort, disability, pain, etc. The present technology can generate surgical plans that include recommended access paths to access target tissue that contributes to the nerve compression. The system can further design instruments that have dimensions, curvatures, and/or features selected based on the patient's anatomy. The instruments can be specifically designed to access and remove target tissue that may contribute or cause nerve compression or other unwanted conditions. In some embodiments, patient-specific kits can include instruments to prepare access paths, alter tissue, and/or remove tissue. The system can produce a kit that is sterilized and provided to the surgical team. The kit can further include one or more implants, access devices (e.g., cannulas, ports, etc.), motorized instruments, and other components for the surgical procedure. The access devices can be custom or patent-specific items.

In some embodiments, the present technology can automatically or at least semi-automatically determine one or more decompression procedures for a subject patient. For example, the computing systems described herein can apply mathematical rules to identify similar patients by analyzing reference patient data sets, and, based on the rules and/or comparison to other patients, can provide recommended decompression procedures that represents the optimal outcome if the subject patient were to undergo surgery. The decompression procedures can be used to treat a wide range of symptoms, conditions, and/or diseases, including, without limitation, spinal nerve compression (e.g., spinal cord compression, spinal nerve root compression, or the like), spinal disc herniation, osteoporosis, stenosis, pinched nerve tissue, or other diseases or conditions. Patient-specific decompressions instruments remove targeted tissue, including, without limitation, bone (e.g., lamina, lateral recesses, facets including the inferior facets, etc.), bone spurs (e.g., bone spurs associated with osteoarthritis), tissue bulging from disks, tissue of thickened ligaments, spinal tumors, displaced tissue (e.g., tissue displaced by a spinal injury), or other tissue that may cause or contribute to spinal nerve compression. In procedures for treating stenosis, the instrument can be used to remove tissue associated with central canal stenosis, lateral recess stenosis, and/or other types of stenosis. The instrument can be viewed using fluoroscope, MR imaging, CT imaging, direct visualization, or the like. In some embodiments, the systems and methods described herein generate a virtual model of the corrected/recommended anatomical configuration (e.g., for surgeon review).

In some embodiments, the present technology can also automatically or at least semi-automatically generate a surgical plan for achieving a previously-identified correction. The surgical correction can decompression correction, spine alignment correction, etc. For example, based off the virtual model of the corrected anatomical configuration, the systems and methods herein can determine a type of surgery (e.g., a decompression and spinal fusion surgery, a decompression and non-fusion surgery, etc.), a surgical approach (e.g., anterior, posterior, etc.), nerve compression sites, targeted tissue contributing to nerve compression, and/or spinal parameters for the corrected anatomical configuration (e.g., lumbar lordosis, Cobb angles, etc.). The surgical plan can be transmitted to a surgeon for review and approval. In some embodiments, the present technology can also design one or more patient-specific implants for achieving the correction via the surgical intervention.

In some embodiments, a computer-readable storage medium stores instructions that, when executed by a computing system, cause a computing system to perform operations. The operations can include receiving patient data for a patient. The patient data can include one or more images of the patient's spinal region showing the patient's anatomy. The computing system can identify, based on the received patient data, at least one site of nerve compression and target tissued at least partially contributing to the nerve compression. An access path for surgically accessing the target tissue can be determined based on the patient's anatomy. The operations can further include designing at least one patient-specific instrument configured to access the target tissue via the access path, perform a decompression step, and/or otherwise alter tissue.

In some embodiments, patient-specific instruments can be designed to perform a series of surgical steps. The present technology can simulate the procedure to develop each of the surgical steps and corresponding instrument designs. This allows the instruments to be designed based on a specific surgical step. In some procedures, for example, an instrument can be a rongeur configured to break apart bone surrounding the spinal canal. Another instrument can include cutting edges, grippers, and other features for removing the loose bone. Another instrument can be a patient-specific reamer configured to gradually remove bone. Yet another instrument can be powered instrument, such as a burr, drill, or rotary tool. The instruments can be configured to produce a resulting geometry that is predictable and patient-specific.

The system can notify the surgical team if an intra-operative decompression procedure (e.g., based on intra-operative imaging, physician input, etc.) varies from a pre-operatively planned decompression procedure, thereby inhibiting or preventing use of subsequent instruments. The surgical team can analyze each surgical step to confirm that one or more criteria are met. In some embodiments, the present technology includes generating, designing, and/or providing patient-specific medical procedures for multiple decompression locations within a patient. The present technology can then design at least two patient-specific instruments for accessing the locations. The patient-specific instruments can each be specifically designed for their respective target region, and thus can have different geometries. In some embodiments, the corrected anatomical configuration of the patient is only achieved by implanting patient-specific implants. In the context of spinal surgery, for example, the present technology may provide a first patient-specific interbody device to be implanted between the L2 and L3 vertebrae, a second patient-specific interbody device to be implanted between the L3 and L4 vertebrae, and a third patient-specific interbody device to be implanted between the L4 and L5 vertebrae. Decompression procedures can be performed at or near L2-L5 vertebrae using the instruments.

In some embodiments, the present technology can predict, model, or simulate disease progression within a particular patient to aid in diagnosis and/or treatment planning. The simulation can be done to model and/or estimate future nerve compression, pain progression, anatomical configurations and/or spine metrics of the patient (a) if no surgical intervention occurs, or (b) for a variety of different surgical intervention options. The progression modeling can thus be used to determine the optimal time for surgical intervention and/or to select which surgical intervention provides the best long-term outcomes. In some embodiments, the disease progression modelling is performed using one or more machine learning models trained based on a plurality of reference patients.

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

The words “comprising,” “having,” “containing,” and “including,” and other forms thereof, are intended to be equivalent in meaning and be open ended in that an item or items following any one of these words is not meant to be an exhaustive listing of such item or items, or meant to be limited to only the listed item or items.

As used herein and in the appended claims, the singular forms “a,” “an,” and “the” include plural references unless the context clearly dictates otherwise.

Although the disclosure herein primarily describes systems and methods for treatment planning in the context of orthopedic surgery, the technology may be applied equally to medical treatment and devices in other fields (e.g., other types of surgical practice). Additionally, although many embodiments herein describe systems and methods with respect to implanted devices, the technology may be applied equally to other types of medical devices (e.g., non-implanted devices).

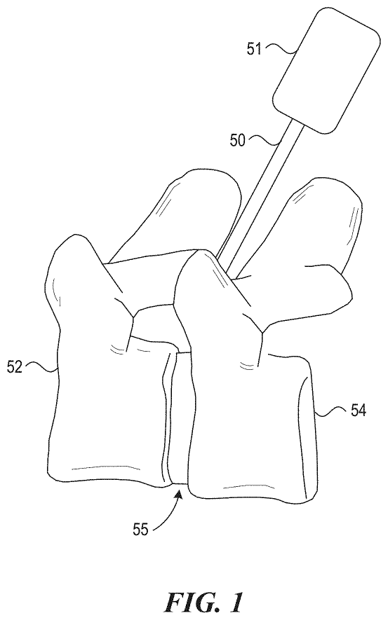

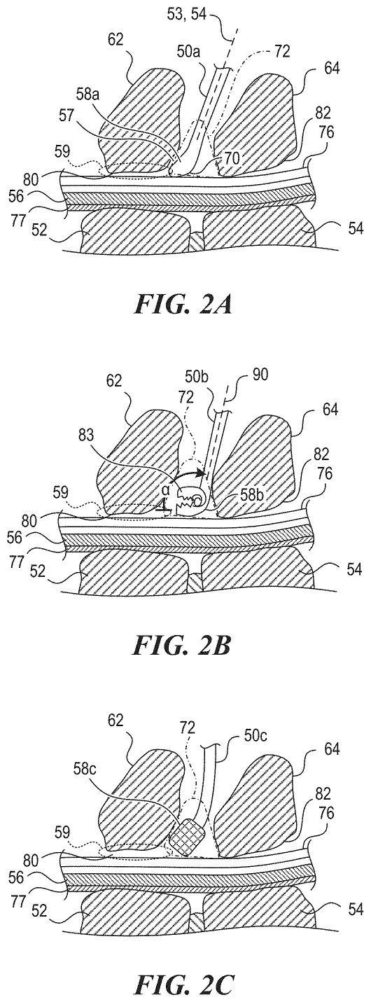

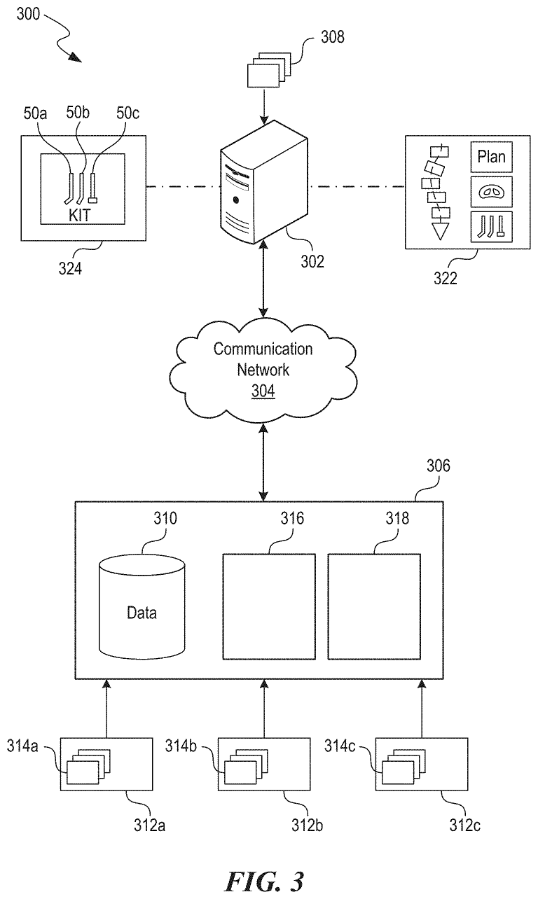

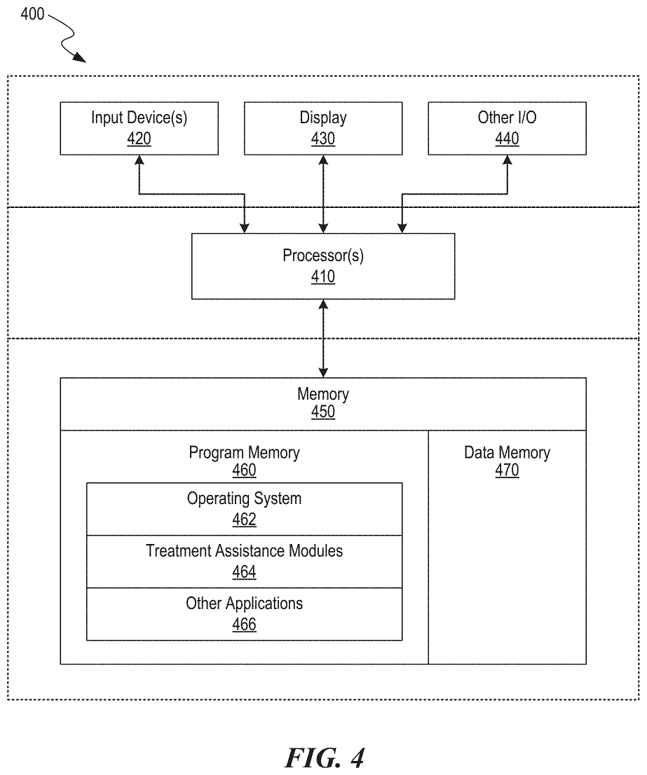

FIG. 1 is a side view of a patient's spine with an instrument 50 positioned to perform at least a portion of a decompression procedure, according to an embodiment. The instrument 50 includes a handle 51 and can be positioned within an access instrument (e.g., cannula, port, etc.) and can be moved to different positions and orientations to allow convenient access to a wide range of sites along the spine. The illustrated decompression instrument 50 is positioned between vertebrae 52, 54 to access a level or region 55 identified with nerve compression. The instrument 50 can be used to remove tissue causing or contributing to the nerve compression. This allows the decompression instrument 50 to be specifically designed to access and then remove target tissue that may contribute or cause nerve compression or other unwanted conditions. For example, the instrument 50 can be replaced with any number of different instruments to treat a wide range of symptoms, conditions, and/or diseases, including, without limitation, spinal nerve compression, spinal disc herniation, osteoporosis, stenosis, or other diseases and conditions. A plurality of decompression instruments can be used to perform a multi-step planned decompression procedure, and instruments can be collectively designed to facilitate ease of use, improve patient outcomes, or the like. The decompression instruments can have an atraumatic surfaces or features configured to atraumatically contact or engage tissue. Example decompression instruments and removal of target tissue 59 as discussed in connection with FIGS. 2A-2C, and the systems discussed in connection with FIGS. 3 and 4 can design surgical kits, decompression instruments, decompression plans, and other patient-specific technology.

FIGS. 2A-2C are detailed side views of distal ends of decompression instruments positioned between vertebrae 52, 54, according to an embodiment. Spinal nerve compression can include spinal cord compression, spinal nerve root compression, or combinations thereof. A spinal cord decompression procedure can include, without limitation, delivering visualization media along the spine, removing bone from one or both vertebrae 52, 54, separating the ligamentum flavum 76 from one or both vertebrae 52, 54, cutting or debulking the ligamentum flavum 76, removing loose tissue, or the like.

FIG. 2A shows the instrument 50a configured to perform steps along the spine without injuring or damaging the spinal cord. The instrument 50a is positioned generally between spinous processes 62, 64 of the vertebrae 52, 54, respectively. The instrument can include a working or distal head 58a configured to contact a targeted zone or tissue 59 identified as contributing or causing nerve compression of, for example, the spinal cord 56. The spinal cord 56 is positioned between the ligamentum flavum 76 and a ligament 77. The spinal cord 56 extends from the brain to the bottom of the spine and extends through vertebral foramen 80, 82, which may be narrowed due to stenosis. The head 58a can have an atraumatic surface 70 configured to atraumatically contact or engage the ligamentum flavum 76 while a cutting edge or roughened surface 57 of head 58a removes bone to enlarge the vertebral foramen 80. In some embodiments, the head 58a is configured to enlarge the neural foramen to reduce or eliminate compression of nerve roots of spinal nerves branching from the spinal cord 56 and exiting the spine to other parts of the body. The configuration and dimensions of the head 58a can be selected based on the tissue to be removed or altered.

FIG. 2B shows an instrument 50b in the form of a rongeur including a distal rongeur head 58b with a jaw 83 designed to fit adjacent to or within the inferior opening of the neural foramen 80 enlarged by the head 58a of FIG. 2A. The dimensions of the head 58b can be selected based on the patient's anatomy and planned prior tissue removal steps (e.g., steps performed using instrument 50a (FIG. 2A)). For example, an angle of the head 58a with respect to a longitudinal axis 90 of the body or shaft can be selected based on the access path for instrument placement.

FIG. 2C shows an instrument 50c in the form of a reamer having a distal cutting or debulking head 58c with an exterior textured surface, spikes, or other features for abrading, cutting, roughening, or otherwise altering tissue. The instrument 50c can include a curved or angled body or shaft that facilitates access to targeted tissue 59. The instruments 50 (FIG. 1), 50a (FIG. 2A), 50b (FIG. 2B) can also have shafts that are straight, angled, curved, etc.

In some procedures, the first group of surgical instruments can be used to perform a decompression procedure, as discussed in connection with FIGS. 2A-2C. The second group of surgical instruments can be used to prepare an implantation site. For example, one or more of the instruments 50a, 50b, and 50c can be used to prepare an intervertebral space for implantation of an intervertebral cage by, for example, removing bony features blocking access to the intervertebral space, modifying (e.g., shaping, texturing, roughening, etc.) the vertebral endplates, etc. An instrument for roughening or smoothing an endplate can have a textured pad (e.g., a circular textured pad) that generally matches the configuration (e.g., topology) of the endplate. The third group of surgical instruments can be used to implant one or more medical devices along the spine. The surgical instruments 50a, 50b, and 50c can be configured to be concurrently or sequentially delivered through an access device (e.g., a cannula, port, etc.) or concurrently or sequentially delivered through multiple access devices (e.g., spaced apart cannulas, ports, etc.). For example, some instruments can be designed to be delivered through a first cannula positioned in the patient and other instruments can be designed to be delivered through a second cannula positioned in the patient. Any number of cannulas, ports, or incisions can be used to insert the instruments. The manufacturing system 324 can include one or more imaging devices used to image the components of the manufactured kit too, for example, confirm the kit is complete. Imaging devices can capture image data for identifying, counting, and inspecting items. The kit can include, without limitation, a container, surgical instruments, instructions for use, and/or other items disclosed herein.

Any number of access devices and/or instruments can be patient-specific to provide one or more benefits disclosed herein. The systems disclosed herein can identify areas or regions of interest (e.g., nerve compression), identify tissue contributing or causing to nerve compression, determine tissue type, access paths (e.g., access paths to areas or regions of interest, paths between tissue, etc.), safe working regions or volumes, or combinations thereof.

FIG. 3 is a network connection diagram illustrating a computing system 300 for providing patient-specific medical care, according to an embodiment. As described in further detail herein, the system 300 is configured to generate a medical treatment plan for a patient. In some embodiments, the system 300 is configured to generate a medical treatment plan for a patient suffering from nerve compression, an orthopedic or spinal disease or disorder, such as trauma (e.g., fractures), cancer, deformity, degeneration, pain (e.g., back pain, leg pain), irregular spinal curvature (e.g., scoliosis, lordosis, kyphosis), irregular spinal displacement (e.g., spondylolisthesis, lateral displacement axial displacement), osteoarthritis, lumbar degenerative disc disease, cervical degenerative disc disease, lumbar spinal stenosis, or cervical spinal stenosis, or a combination thereof. The medical treatment plan can include surgical information, surgical plans, technology recommendations (e.g., device and/or instrument recommendations), and/or medical device designs. For example, the medical treatment plan can include at least one treatment procedure (e.g., a surgical procedure or intervention) and/or at least one medical device (e.g., an implanted medical device (also referred to herein as an “implant” or “implanted device”) or implant delivery instrument).

In some embodiments, the system 300 generates a medical treatment plan that is customized for a particular patient or group of patients, also referred to herein as a “patient-specific” or “personalized” treatment plan. The patient-specific treatment plan can include at least one patient-specific surgical procedure and/or at least one patient-specific medical device that are designed and/or optimized for the patient's particular characteristics (e.g., condition, anatomy, pathology, condition, medical history). For example, the patient-specific medical device can be designed and manufactured specifically for the particular patient, rather than being an off-the-shelf device. However, it shall be appreciated that a patient-specific treatment plan can also include aspects that are not customized for the particular patient. For example, a patient-specific or personalized surgical procedure can include one or more instructions, portions, steps, etc. that are non-patient-specific. Likewise, a patient-specific or personalized medical device can include one or more components that are non-patient-specific, and/or can be used with an instrument or tool that is non-patient-specific. Personalized implant designs can be used to manufacture or select patient-specific technologies, including medical devices, instruments, and/or surgical kits. For example, a personalized surgical kit can include one or more patient-specific devices, patient-specific instruments, non-patient-specific technology (e.g., standard instruments, devices, etc.), instructions for use, patient-specific treatment plan information, or a combination thereof. In some embodiments, the personalized surgical kit can include a series of patient-specific instruments designed to perform surgical steps. For example, a first group of surgical instruments can be used to perform a decompression procedure. A second group of surgical instruments can be used to prepare an implantation site. A third group of surgical instruments can be used to implant one or more medical devices along the spine. The medical devices can be, for example, intervertebral bodies, interspinous spacers, pedicle screw/rod systems, or other medical devices disclosed herein.

The system 300 can design a surgical kit for a surgical plan. The system can determine specific steps for the surgical plan. The system can then design one or more items for each step. The surgical kit can include, without limitation, any number of instruments, implants, or other items disclosed herein. The instruments can be designed based on the implants. Additionally or alternatively, the implants can be designed based on the instruments. This design flexibility can provide for patient-specific treatment with instrument/device compatibility, thereby enhancing outcomes. The system 300 can send instructions to manufacture kits and validate the manufactured kits are complete. Imaging devices can capture image data for identifying, counting, and inspecting items in the kit. Example components of surgical kits are discussed in connection with FIGS. 1-3, 14 and 16A-16C.

The system 300 includes a client computing device 302, which can be a user device, such as a smart phone, mobile device, laptop, desktop, personal computer, tablet, phablet, or other such devices known in the art. As discussed further herein, the client computing device 302 can include one or more processors, and memory storing instructions executable by the one or more processors to perform the methods described herein. The client computing device 302 can be associated with a healthcare provider that is treating the patient. Although FIG. 3 illustrates a single client computing device 302, in alternative embodiments, the client computing device 302 can instead be implemented as a client computing system encompassing a plurality of computing devices, such that the operations described herein with respect to the client computing device 302 can instead be performed by the computing system and/or the plurality of computing devices.

The client computing device 302 is configured to receive a patient data set 308 associated with a patient to be treated. The patient data set 308 can include data representative of the patient's condition, anatomy, pathology, medical history, preferences, and/or any other information or parameters relevant to the patient. For example, the patient data set 308 can include medical history, surgical intervention data, treatment outcome data, progress data (e.g., physician notes), patient feedback (e.g., feedback acquired using quality of life questionnaires, surveys), clinical data, provider information (e.g., physician, hospital, surgical team), patient information (e.g., demographics, sex, age, height, weight, type of pathology, occupation, activity level, tissue information, health rating, comorbidities, health related quality of life (HRQL)), vital signs, diagnostic results, medication information, allergies, image data (e.g., camera images, Magnetic Resonance Imaging (MRI) images, ultrasound images, Computerized Aided Tomography (CAT) scan images, Positron Emission Tomography (PET) images, X-Ray images), diagnostic equipment information (e.g., manufacturer, model number, specifications, user-selected settings/configurations, etc.), or the like. In some embodiments, the patient data set 308 includes data representing one or more of patient identification number (ID), age, gender, body mass index (BMI), lumbar lordosis, Cobb angle(s), pelvic incidence, disc height, segment flexibility, bone quality, rotational displacement, and/or treatment level of the spine.

The client computing device 302 is operably connected via a communication network 304 to a server 306, thus allowing for data transfer between the client computing device 302 and the server 306. The communication network 304 may be a wired and/or a wireless network. The communication network 304, if wireless, may be implemented using communication techniques such as Visible Light Communication (VLC), Worldwide Interoperability for Microwave Access (WiMAX), Long term evolution (LTE), Wireless local area network (WLAN), Infrared (IR) communication, Public Switched Telephone Network (PSTN), Radio waves, and/or other communication techniques known in the art.

The server 306, which may also be referred to as a “treatment assistance network” or “prescriptive analytics network,” can include one or more computing devices and/or systems. As discussed further herein, the server 306 can include one or more processors, and memory storing instructions executable by the one or more processors to perform the methods described herein. In some embodiments, the server 306 is implemented as a distributed “cloud” computing system or facility across any suitable combination of hardware and/or virtual computing resources.

The client computing device 302 and server 306 can individually or collectively perform the various methods described herein for providing patient-specific medical care. For example, some or all of the steps of the methods described herein can be performed by the client computing device 302 alone, the server 306 alone, or a combination of the client computing device 302 and the server 306. Thus, although certain operations are described herein with respect to the server 306, it shall be appreciated that these operations can also be performed by the client computing device 302, and vice-versa.

The server 306 includes at least one database 310 configured to store reference data useful for the treatment planning methods described herein. The reference data can include historical and/or clinical data from the same or other patients, data collected from prior surgeries and/or other treatments of patients by the same or other healthcare providers, data relating to medical device designs, data collected from study groups or research groups, data from practice databases, data from academic institutions, data from implant manufacturers or other medical device manufacturers, data from imaging studies, data from simulations, clinical trials, demographic data, treatment data, outcome data, mortality rates, or the like.

In some embodiments, the database 310 includes a plurality of reference patient data sets, each patient reference data set associated with a corresponding reference patient. For example, the reference patient can be a patient that previously received treatment or is currently receiving treatment. Each reference patient data set can include data representative of the corresponding reference patient's condition, anatomy, pathology, medical history, disease progression, preferences, and/or any other information or parameters relevant to the reference patient, such as any of the data described herein with respect to the patient data set 308. In some embodiments, the reference patient data set includes pre-operative data, intra-operative data, and/or post-operative data. For example, a reference patient data set can include data representing one or more of patient ID, age, gender, BMI, lumbar lordosis, Cobb angle(s), pelvic incidence, disc height, segment flexibility, bone quality, rotational displacement, and/or treatment level of the spine. As another example, a reference patient data set can include treatment data regarding at least one treatment procedure performed on the reference patient, such as descriptions of surgical procedures or interventions (e.g., surgical approaches, bony resections, surgical maneuvers, corrective maneuvers, placement of implants or other devices). In some embodiments, the treatment data includes medical device design data for at least one medical device used to treat the reference patient, such as physical properties (e.g., size, shape, volume, material, mass, weight), mechanical properties (e.g., stiffness, strength, modulus, hardness), and/or biological properties (e.g., osteo-integration, cellular adhesion, anti-bacterial properties, anti-viral properties). In yet another example, a reference patient data set can include outcome data representing an outcome of the treatment of the reference patient, such as corrected anatomical metrics, presence of fusion, HRQL, activity level, return to work, complications, recovery times, efficacy, mortality, and/or follow-up surgeries.

In some embodiments, the server 306 receives at least some of the reference patient data sets from a plurality of healthcare provider computing systems (e.g., systems 312a-312c, collectively 312). The server 306 can be connected to the healthcare provider computing systems 312 via one or more communication networks (not shown). Each healthcare provider computing system 312 can be associated with a corresponding healthcare provider (e.g., physician, surgeon, medical clinic, hospital, healthcare network, etc.). Each healthcare provider computing system 312 can include at least one reference patient data set (e.g., reference patient data sets 314a-314c, collectively 314) associated with reference patients treated by the corresponding healthcare provider. The reference patient data sets 314 can include, for example, electronic medical records, electronic health records, biomedical data sets, etc. The reference patient data sets 314 can be received by the server 306 from the healthcare provider computing systems 312 and can be reformatted into different formats for storage in the database 310. Optionally, the reference patient data sets 314 can be processed (e.g., cleaned) to ensure that the represented patient parameters are likely to be useful in the treatment planning methods described herein.

As described in further detail herein, the server 306 can be configured with one or more algorithms that generate patient-specific treatment plan data (e.g., treatment procedures, medical devices) based on the reference data. In some embodiments, the patient-specific data is generated based on correlations between the patient data set 308 and the reference data. Optionally, the server 306 can predict outcomes, including recovery times, efficacy based on clinical end points, likelihood of success, predicted mortality, predicted related follow-up surgeries, or the like. In some embodiments, the server 306 can continuously or periodically analyze patient data (including patient data obtained during the patient stay) to determine near real-time or real-time risk scores, mortality prediction, etc.

In some embodiments, the server 306 includes one or more modules for performing one or more steps of the patient-specific treatment planning methods described herein. For example, in the depicted embodiment, the server 306 includes a data analysis module 316 and a treatment planning module 318. In alternative embodiments, one or more of these modules may be combined with each other, or may be omitted. Thus, although certain operations are described herein with respect to a particular module or modules, this is not intended to be limiting, and such operations can be performed by a different module or modules in alternative embodiments.

The data analysis module 316 is configured with one or more algorithms for identifying a subset of reference data from the database 310 that is likely to be useful in developing a patient-specific treatment plan. For example, the data analysis module 316 can compare patient-specific data (e.g., the patient data set 308 received from the client computing device 302) to the reference data from the database 310 (e.g., the reference patient data sets) to identify similar data (e.g., one or more similar patient data sets in the reference patient data sets). The comparison can be based on one or more parameters, such as age, gender, BMI, lumbar lordosis, pelvic incidence, and/or treatment levels. The parameter(s) can be used to calculate a similarity score for each reference patient. The similarity score can represent a statistical correlation between the patient data set 308 and the reference patient data set. Accordingly, similar patients can be identified based on whether the similarity score is above, below, or at a specified threshold value. For example, as described in greater detail below, the comparison can be performed by assigning values to each parameter and determining the aggregate difference between the subject patient and each reference patient. Reference patients whose aggregate difference is below a threshold can be considered to be similar patients.

The data analysis module 316 can further be configured with one or more algorithms to select a subset of the reference patient data sets, e.g., based on similarity to the patient data set 308 and/or treatment outcome of the corresponding reference patient. For example, the data analysis module 316 can identify one or more similar patient data sets in the reference patient data sets, and then select a subset of the similar patient data sets based on whether the similar patient data set includes data indicative of a favorable or desired treatment outcome. The outcome data can include data representing one or more outcome parameters, such as corrected anatomical metrics, presence of fusion, HRQL, activity level, complications, recovery times, efficacy, mortality, or follow-up surgeries. As described in further detail below, in some embodiments, the data analysis module 316 calculates an outcome score by assigning values to each outcome parameter. A patient can be considered to have a favorable outcome if the outcome score is above, below, or at a specified threshold value.

In some embodiments, the data analysis module 316 selects a subset of the reference patient data sets based at least in part on user input (e.g., from a clinician, surgeon, physician, healthcare provider). For example, the user input can be used in identifying similar patient data sets. In some embodiments, weighting of similarity and/or outcome parameters can be selected by a healthcare provider or physician to adjust the similarity and/or outcome score based on clinician input. In further embodiments, the healthcare provider or physician can select the set of similarity and/or outcome parameters (or define new similarity and/or outcome parameters) used to generate the similarity and/or outcome score, respectively.

In some embodiments, the data analysis module 316 includes one or more algorithms used to select a set or subset of the reference patient data sets based on criteria other than patient parameters. For example, the one or more algorithms can be used to select the subset based on healthcare provider parameters (e.g., based on healthcare provider ranking/scores such as hospital/physician expertise, number of procedures performed, hospital ranking, etc.) and/or healthcare resource parameters (e.g., diagnostic equipment, facilities, surgical equipment such as surgical robots), or other non-patient related information that can be used to predict outcomes and risk profiles for procedures for the present healthcare provider. For example, reference patient data sets with images captured from similar diagnostic equipment can be aggregated to reduce or limit irregularities due to variation between diagnostic equipment. Additionally, patient-specific treatment plans can be developed for a particular health-care provider using data from similar healthcare providers (e.g., healthcare providers with traditionally similar outcomes, physician expertise, surgical teams, etc.). In some embodiments, reference healthcare provider data sets, hospital data sets, physician data sets, surgical team data sets, post-treatment data set, and other data sets can be utilized. By way of example, a patient-specific treatment plan to perform a battlefield surgery can be based on reference patient data from similar battlefield surgeries and/or data sets associated with battlefield surgeries. In another example, the patient-specific treatment plan can be generated based on available robotic surgical systems. The reference patient data sets can be selected based on patients that have been operated on using comparable robotic surgical systems under similar conditions (e.g., size and capabilities of surgical teams, hospital resources, etc.).

The treatment planning module 318 is configured with one or more algorithms to generate at least one treatment plan (e.g., pre-operative plans, surgical plans, post-operative plans etc.) based on the output from the data analysis module 316. In some embodiments, the treatment planning module 318 is configured to develop and/or implement at least one predictive model for generating the patient-specific treatment plan, also known as a “prescriptive model.” The predictive model(s) can be developed using clinical knowledge, statistics, machine learning, AI, neural networks, or the like. In some embodiments, the output from the data analysis module 316 is analyzed (e.g., using statistics, machine learning, neural networks, AI) to identify correlations between data sets, patient parameters, healthcare provider parameters, healthcare resource parameters, treatment procedures, medical device designs, and/or treatment outcomes. These correlations can be used to develop at least one predictive model that predicts the likelihood that a treatment plan will produce a favorable outcome for the particular patient. The predictive model(s) can be validated, e.g., by inputting data into the model(s) and comparing the output of the model to the expected output.

In some embodiments, the treatment planning module 318 is configured to generate the treatment plan based on previous treatment data from reference patients. For example, the treatment planning module 318 can receive a selected subset of reference patient data sets and/or similar patient data sets from the data analysis module 316, and determine or identify treatment data from the selected subset. The treatment data can include, for example, treatment procedure data (e.g., surgical procedure or intervention data) and/or medical device design data (e.g., implant design data) that are associated with favorable or desired treatment outcomes for the corresponding patient. The treatment planning module 318 can analyze the treatment procedure data and/or medical device design data to determine an optimal treatment protocol for the patient to be treated. For example, the treatment procedures and/or medical device designs can be assigned values and aggregated to produce a treatment score. The patient-specific treatment plan can be determined by selecting treatment plan(s) based on the score (e.g., higher or highest score; lower or lowest score; score that is above, below, or at a specified threshold value). The personalized patient-specific treatment plan can be based on, at least in part, the patient-specific technologies or patient-specific selected technology.

Alternatively or in combination, the treatment planning module 318 can generate the treatment plan based on correlations between data sets. For example, the treatment planning module 318 can correlate treatment procedure data and/or medical device design data from similar patients with favorable outcomes (e.g., as identified by the data analysis module 316). Correlation analysis can include transforming correlation coefficient values to values or scores. The values/scores can be aggregated, filtered, or otherwise analyzed to determine one or more statistical significances. These correlations can be used to determine treatment procedure(s) and/or medical device design(s) that are optimal or likely to produce a favorable outcome for the patient to be treated.

Alternatively or in combination, the treatment planning module 318 can generate the treatment plan using one or more AI techniques. AI techniques can be used to develop computing systems capable of simulating aspects of human intelligence, e.g., learning, reasoning, planning, problem solving, decision making, etc. AI techniques can include, but are not limited to, case-based reasoning, rule-based systems, artificial neural networks, decision trees, support vector machines, regression analysis, Bayesian networks (e.g., naïve Bayes classifiers), genetic algorithms, cellular automata, fuzzy logic systems, multi-agent systems, swarm intelligence, data mining, machine learning (e.g., supervised learning, unsupervised learning, reinforcement learning), and hybrid systems.

In some embodiments, the treatment planning module 318 generates the treatment plan using one or more trained machine learning models. Various types of machine learning models, algorithms, and techniques are suitable for use with the present technology. In some embodiments, the machine learning model is initially trained on a training data set, which is a set of examples used to fit the parameters (e.g., weights of connections between “neurons” in artificial neural networks) of the model. For example, the training data set can include any of the reference data stored in database 310, such as a plurality of reference patient data sets or a selected subset thereof (e.g., a plurality of similar patient data sets).

In some embodiments, the machine learning model (e.g., a neural network or a naïve Bayes classifier) may be trained on the training data set using a supervised learning method (e.g., gradient descent or stochastic gradient descent). The training data set can include pairs of generated “input vectors” with the associated corresponding “answer vector” (commonly denoted as the target). The current model is run with the training data set and produces a result, which is then compared with the target, for each input vector in the training data set. Based on the result of the comparison and the specific learning algorithm being used, the parameters of the model are adjusted. The model fitting can include both variable selection and parameter estimation. The fitted model can be used to predict the responses for the observations in a second data set called the validation data set. The validation data set can provide an unbiased evaluation of a model fit on the training data set while tuning the model parameters. Validation data sets can be used for regularization by early stopping, e.g., by stopping training when the error on the validation data set increases, as this may be a sign of overfitting to the training data set. In some embodiments, the error of the validation data set error can fluctuate during training, such that ad-hoc rules may be used to decide when overfitting has truly begun. Finally, a test data set can be used to provide an unbiased evaluation of a final model fit on the training data set.

To generate a treatment plan, the patient data set 308 can be input into the trained machine learning model(s). Additional data, such as the selected subset of reference patient data sets and/or similar patient data sets, and/or treatment data from the selected subset, can also be input into the trained machine learning model(s). The trained machine learning model(s) can then calculate whether various candidate treatment procedures and/or medical device designs are likely to produce a favorable outcome for the patient. Based on these calculations, the trained machine learning model(s) can select at least one treatment plan for the patient. In embodiments where multiple trained machine learning models are used, the models can be run sequentially or concurrently to compare outcomes and can be periodically updated using training data sets. The treatment planning module 318 can use one or more of the machine learning models based the model's predicted accuracy score.

The patient-specific treatment plan generated by the treatment planning module 318 can include at least one patient-specific treatment procedure (e.g., a surgical procedure or intervention) and/or at least one patient-specific medical device (e.g., an implant or implant delivery instrument). A patient-specific treatment plan can include an entire surgical procedure or portions thereof. Additionally, one or more patient-specific medical devices can be specifically selected or designed for the corresponding surgical procedure, thus allowing for the various components of the patient-specific technology to be used in combination to treat the patient.

In some embodiments, the patient-specific treatment procedure includes an orthopedic surgery procedure, such as spinal surgery, hip surgery, knee surgery, jaw surgery, hand surgery, shoulder surgery, elbow surgery, total joint reconstruction (arthroplasty), skull reconstruction, foot surgery, or ankle surgery. Spinal surgery can include spinal fusion surgery, such as posterior lumbar interbody fusion (PLIF), anterior lumbar interbody fusion (ALIF), transverse or transforaminal lumbar interbody fusion (TLIF), lateral lumbar interbody fusion (LLIF), direct lateral lumbar interbody fusion (DLIF), or extreme lateral lumbar interbody fusion (XLIF). In some embodiments, the patient-specific treatment procedure includes descriptions of and/or instructions for performing one or more aspects of a patient-specific surgical procedure. For example, the patient-specific surgical procedure can include one or more of a surgical approach, a corrective maneuver, a bony resection, or implant placement.

In some embodiments, the patient-specific medical device design includes a design for an orthopedic implant and/or a design for an instrument for delivering an orthopedic implant. Examples of such implants include, but are not limited to, screws (e.g., bone screws, spinal screws, pedicle screws, facet screws), interbody implant devices (e.g., intervertebral implants), cages, plates, rods, disks, fusion devices, spacers, rods, expandable devices, stents, brackets, ties, scaffolds, fixation device, anchors, nuts, bolts, rivets, connectors, tethers, fasteners, joint replacements, hip implants, or the like. Examples of instruments include, but are not limited to, screw guides, cannulas, ports, catheters, insertion tools, or the like.

A patient-specific medical device design can include data representing one or more of physical properties (e.g., size, shape, volume, material, mass, weight), mechanical properties (e.g., stiffness, strength, modulus, hardness), and/or biological properties (e.g., osteo-integration, cellular adhesion, anti-bacterial properties, anti-viral properties) of a corresponding medical device. For example, a design for an orthopedic implant can include implant shape, size, material, and/or effective stiffness (e.g., lattice density, number of struts, location of struts, etc.). In some embodiments, the generated patient-specific medical device design is a design for an entire device. Alternatively, the generated design can be for one or more components of a device, rather than the entire device.

In some embodiments, the design is for one or more patient-specific device components that can be used with standard, off-the-shelf components. For example, in a spinal surgery, a pedicle screw kit can include both standard components and patient-specific customized components. In some embodiments, the generated design is for a patient-specific medical device that can be used with a standard, off-the-shelf delivery instrument. For example, the implants (e.g., screws, screw holders, rods) can be designed and manufactured for the patient, while the instruments for delivering the implants can be standard instruments. This approach allows the components that are implanted to be designed and manufactured based on the patient's anatomy and/or surgeon's preferences to enhance treatment. The patient-specific devices described herein are expected to improve delivery into the patient's body, placement at the treatment site, and/or interaction with the patient's anatomy.

In embodiments where the patient-specific treatment plan includes a surgical procedure to implant a medical device, the treatment planning module 318 can also store various types of implant surgery information, such as implant parameters (e.g., types, dimensions), availability of implants, aspects of a pre-operative plan (e.g., initial implant configuration, detection and measurement of the patient's anatomy, etc.), FDA requirements for implants (e.g., specific implant parameters and/or characteristics for compliance with FDA regulations), or the like. In some embodiments, the treatment planning module 318 can convert the implant surgery information into formats useable for machine-learning based models and algorithms. For example, the implant surgery information can be tagged with particular identifiers for formulas or can be converted into numerical representations suitable for supplying to the trained machine learning model(s). The treatment planning module 318 can also store information regarding the patient's anatomy, such as two- or three-dimensional images or models of the anatomy, and/or information regarding the biology, geometry, and/or mechanical properties of the anatomy. The anatomy information can be used to inform implant design and/or placement.

The treatment plan(s) generated by the treatment planning module 318 can be transmitted via the communication network 304 to the client computing device 302 for output to a user (e.g., clinician, surgeon, healthcare provider, patient). In some embodiments, the client computing device 302 includes or is operably coupled to a display 322 for outputting the treatment plan(s). The display 322 can include a graphical user interface (GUI) for visually depicting various aspects of the treatment plan(s). For example, the display 322 can show various aspects of a surgical procedure to be performed on the patient, such as the surgical approach, treatment levels, corrective maneuvers, tissue resection, and/or implant placement. To facilitate visualization, a virtual model of the surgical procedure can be displayed. As another example, the display 322 can show a design for a medical device to be implanted in the patient, such as a two- or three-dimensional model of the device design. The display 322 can also show patient information, such as two- or three-dimensional images or models of the patient's anatomy where the surgical procedure is to be performed and/or where the device is to be implanted. The client computing device 302 can further include one or more user input devices (not shown) allowing the user to modify, select, approve, and/or reject the displayed treatment plan(s).

In some embodiments, the medical device design(s) generated by the treatment planning module 318 can be transmitted from the client computing device 302 and/or server 306 to a manufacturing system 324 for manufacturing a corresponding kit (e.g., illustrated decompression kit), medical device, etc. The manufacturing system 324 can be located on site or off site. On-site manufacturing can reduce the number of sessions with a patient and/or the time to be able to perform the surgery whereas off-site manufacturing can be useful make the complex devices. Off-site manufacturing facilities can have specialized manufacturing equipment. In some embodiments, more complicated device components can be manufactured off site, while simpler device components can be manufactured on site. The manufacturing system 324 can include manufacturing subsystems capable of manufacturing components. In some embodiments, a first subsystem can be configured to manufacture one or more patient-specific instruments, as discussed in connection with FIGS. 1-2C. Another subsystem can be used to manufacture a medical implant, such as an intervertebral cage. The manufacturing capabilities of the subsystems can be selected based on the instruments to be manufactured.

Various types of manufacturing systems are suitable for use in accordance with the embodiments herein. For example, the manufacturing system 324 can be configured for additive manufacturing, such as three-dimensional (3D) printing, stereolithography (SLA), digital light processing (DLP), fused deposition modeling (FDM), selective laser sintering (SLS), selective laser melting (SLM), selective heat sintering (SHM), electronic beam melting (EBM), laminated object manufacturing (LOM), powder bed printing (PP), thermoplastic printing, direct material deposition (DMD), inkjet photo resin printing, or like technologies, or combination thereof. Alternatively or in combination, the manufacturing system 324 can be configured for subtractive (traditional) manufacturing, such as CNC machining, electrical discharge machining (EDM), grinding, laser cutting, water jet machining, manual machining (e.g., milling, lathe/turning), or like technologies, or combinations thereof. The manufacturing system 324 can manufacture one or more patient-specific medical devices based on fabrication instructions or data (e.g., CAD data, 3D data, digital blueprints, stereolithography data, or other data suitable for the various manufacturing technologies described herein). Different components of the system 300 can generate at least a portion of the manufacturing data used by the manufacturing system 324. The manufacturing data can include, without limitation, fabrication instructions (e.g., programs executable by additive manufacturing equipment, subtractive manufacturing equipment, etc.), 3D data, CAD data (e.g., CAD files), CAM data (e.g., CAM files), path data (e.g., print head paths, tool paths, etc.), material data, tolerance data, surface finish data (e.g., surface roughness data), regulatory data (e.g., FDA requirements, reimbursement data, etc.), or the like. The manufacturing system 324 can analyze the manufacturability of the implant design based on the received manufacturing data. The implant design can be finalized by altering geometries, surfaces, etc. and then generating manufacturing instructions. In some embodiments, the server 306 generates at least a portion of the manufacturing data, which is transmitted to the manufacturing system 324.

The manufacturing system 324 can generate CAM data, print data (e.g., powder bed print data, thermoplastic print data, photo resin data, etc.), or the like and can include additive manufacturing equipment, subtractive manufacturing equipment, thermal processing equipment, or the like. The additive manufacturing equipment can be 3D printers, stereolithography devices, digital light processing devices, fused deposition modeling devices, selective laser sintering devices, selective laser melting devices, electronic beam melting devices, laminated object manufacturing devices, powder bed printers, thermoplastic printers, direct material deposition devices, or inkjet photo resin printers, or like technologies. The subtractive manufacturing equipment can be CNC machines, electrical discharge machines, grinders, laser cutters, water jet machines, manual machines (e.g., milling machines, lathes, etc.), or like technologies. Both additive and subtractive techniques can be used to produce implants with complex geometries, surface finishes, material properties, etc. The generated fabrication instructions can be configured to cause the manufacturing system 324 to manufacture the patient-specific orthopedic implant that matches or is therapeutically the same as the patient-specific design. In some embodiments, the patient-specific medical device can include features, materials, and designs shared across designs to simplify manufacturing. For example, deployable patient-specific medical devices for different patients can have similar internal deployment mechanisms but have different deployed configurations. In some embodiments, the components of the patient-specific medical devices are selected from a set of available pre-fabricated components and the selected pre-fabricated components can be modified based on the fabrication instructions or data.

The treatment plans described herein can be performed by a surgeon, a surgical robot, or a combination thereof, thus allowing for treatment flexibility. In some embodiments, the surgical procedure can be performed entirely by a surgeon, entirely by a surgical robot, or a combination thereof. For example, one step of a surgical procedure can be manually performed by a surgeon and another step of the procedure can be performed by a surgical robot. In some embodiments the treatment planning module 318 generates control instructions configured to cause a surgical robot (e.g., robotic surgery systems, navigation systems, etc.) to partially or fully perform a surgical procedure. The control instructions can be transmitted to the robotic apparatus by the client computing device 302 and/or the server 306.

Following the treatment of the patient in accordance with the treatment plan, treatment progress can be monitored over one or more time periods to update the data analysis module 316 and/or treatment planning module 318. Post-treatment data can be added to the reference data stored in the database 310. The post-treatment data can be used to train machine learning models for developing patient-specific treatment plans, patient-specific medical devices, or combinations thereof.

It shall be appreciated that the components of the system 300 can be configured in many different ways. For example, in alternative embodiments, the database 310, the data analysis module 316 and/or the treatment planning module 318 can be components of the client computing device 302, rather than the server 306. As another example, the database 310 the data analysis module 316, and/or the treatment planning module 318 can be located across a plurality of different servers, computing systems, or other types of cloud-computing resources, rather than at a single server 306 or client computing device 302.

Additionally, in some embodiments, the system 300 can be operational with numerous other computing system environments or configurations. Examples of computing systems, environments, and/or configurations that may be suitable for use with the technology include, but are not limited to, personal computers, server computers, handheld or laptop devices, cellular telephones, wearable electronics, tablet devices, multiprocessor systems, microprocessor-based systems, programmable consumer electronics, network PCs, minicomputers, mainframe computers, distributed computing environments that include any of the above systems or devices, or the like.

FIG. 4 illustrates a computing device 400 suitable for use in connection with the system 300 of FIG. 3, according to an embodiment. The computing device 400 can be incorporated in various components of the system 300 of FIG. 3, such as the client computing device 302 or the server 306. The computing device 400 includes one or more processors 410 (e.g., CPU(s), GPU(s), HPU(s), etc.). The processor(s) 410 can be a single processing unit or multiple processing units in a device or distributed across multiple devices. The processor(s) 410 can be coupled to other hardware devices, for example, with the use of a bus, such as a PCI bus or SCSI bus. The processor(s) 410 can be configured to execute one more computer-readable program instructions, such as program instructions to carry out of any of the methods described herein.

The computing device 400 can include one or more input devices 420 that provide input to the processor(s) 410, e.g., to notify it of actions from a user of the device 400. The actions can be mediated by a hardware controller that interprets the signals received from the input device and communicates the information to the processor(s) 410 using a communication protocol. Input device(s) 420 can include, for example, a mouse, a keyboard, a touchscreen, an infrared sensor, a touchpad, a wearable input device, a camera- or image-based input device, a microphone, or other user input devices.

The computing device 400 can include a display 430 used to display various types of output, such as text, models, virtual procedures, surgical plans, implants, graphics, and/or images (e.g., images with voxels indicating radiodensity units or Hounsfield units representing the density of the tissue at a location). In some embodiments, the display 430 provides graphical and textual visual feedback to a user. The processor(s) 410 can communicate with the display 430 via a hardware controller for devices. In some embodiments, the display 430 includes the input device(s) 420 as part of the display 430, such as when the input device(s) 420 include a touchscreen or is equipped with an eye direction monitoring system. In alternative embodiments, the display 430 is separate from the input device(s) 420. Examples of display devices include an LCD display screen, an LED display screen, a projected, holographic, or augmented reality display (e.g., a heads-up display device or a head-mounted device), and so on.

Optionally, other I/O devices 440 can also be coupled to the processor(s) 410, such as a network card, video card, audio card, USB, firewire or other external device, camera, printer, speakers, CD-ROM drive, DVD drive, disk drive, or Blu-Ray device. Other I/O devices 440 can also include input ports for information from directly connected medical equipment such as imaging apparatuses, including MRI machines, X-Ray machines, CT machines, etc. Other I/O devices 440 can further include input ports for receiving data from these types of machine from other sources, such as across a network or from previously captured data, for example, stored in a database.

In some embodiments, the computing device 400 also includes a communication device (not shown) capable of communicating wirelessly or wire-based with a network node. The communication device can communicate with another device or a server through a network using, for example, TCP/IP protocols. The computing device 400 can utilize the communication device to distribute operations across multiple network devices, including imaging equipment, manufacturing equipment, etc.

The computing device 400 can include memory 450, which can be in a single device or distributed across multiple devices. Memory 450 includes one or more of various hardware devices for volatile and non-volatile storage, and can include both read-only and writable memory. For example, a memory can comprise random access memory (RAM), various caches, CPU registers, read-only memory (ROM), and writable non-volatile memory, such as flash memory, hard drives, floppy disks, CDs, DVDs, magnetic storage devices, tape drives, device buffers, and so forth. A memory is not a propagating signal divorced from underlying hardware; a memory is thus non-transitory. In some embodiments, the memory 450 is a non-transitory computer-readable storage medium that stores, for example, programs, software, data, or the like. In some embodiments, memory 450 can include program memory 460 that stores programs and software, such as an operating system 462, one or more treatment assistance modules 464, and other application programs 466. The treatment assistance module(s) 464 can include one or more modules configured to perform the various methods described herein (e.g., the data analysis module 316 and/or treatment planning module 318 described with respect to FIG. 3). Memory 450 can also include data memory 470 that can include, e.g., reference data, configuration data, settings, user options or preferences, etc., which can be provided to the program memory 460 or any other element of the computing device 400.

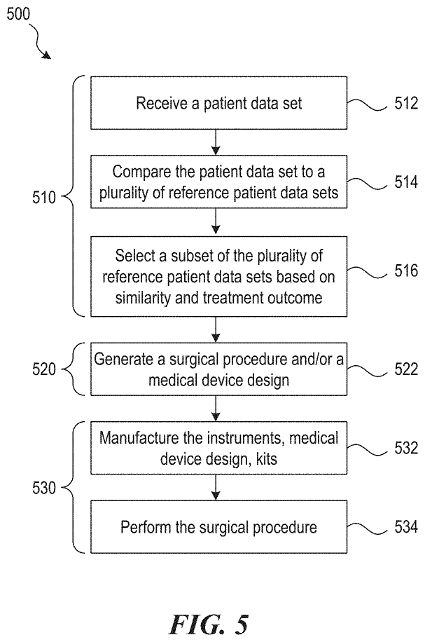

FIG. 5 is a flow diagram illustrating a method 500 for providing patient-specific medical care, according to an embodiment. The method 500 can include a data phase 510, a modeling phase 520, and an execution phase 530. The data phase 510 can include collecting data of a patient to be treated (e.g., pathology data), and comparing the patient data to reference data (e.g., prior patient data such as pathology, surgical, and/or outcome data). For example, a patient data set can be received (block 512). The patient data set can be compared to a plurality of reference patient data sets (block 514), e.g., in order to identify one or more similar patient data sets in the plurality of reference patient data sets. Each of the plurality of reference patient data sets can include data representing one or more of age, gender, BMI, lumbar lordosis, Cobb angle(s), pelvic incidence, disc height, segment flexibility, bone quality, rotational displacement, or treatment level of the spine.

A subset of the plurality of reference patient data sets can be selected (block 516), e.g., based on similarity to the patient data set and/or treatment outcomes of the corresponding reference patients. For example, a similarity score can be generated for each reference patient data set, based on the comparison of the patient data set and the reference patient data set. The similarity score can represent a statistical correlation between the patient data and the reference patient data set. One or more similar patient data sets can be identified based, at least partly, on the similarity score.

In some embodiments, each patient data set of the selected subset includes and/or is associated with data indicative of a favorable treatment outcome (e.g., a favorable treatment outcome based on a single target outcome, aggregate outcome score, outcome thresholding). The data can include, for example, data representing one or more of corrected anatomical metrics, presence of fusion, health related quality of life, activity level, or complications. In some embodiments, the data is or includes an outcome score, which can be calculated based on a single target outcome, an aggregate outcome, and/or an outcome threshold.

Optionally, the data analysis phase 510 can include identifying or determining, for at least one patient data set of the selected subset (e.g., for at least one similar patient data set), surgical procedure data and/or medical device design data associated with the favorable treatment outcome. The surgical procedure data can include data representing one or more of a surgical approach, a corrective maneuver, a bony resection, or implant placement. The at least one medical device design can include data representing one or more of physical properties, mechanical properties, or biological properties of a corresponding medical device. In some embodiments, the at least one patient-specific medical device design includes a design for an implant or an implant delivery instrument.

In the modeling phase 520, a surgical procedure and/or medical device design is generated (block 522). The generating step can include developing at least one predictive model based on the patient data set and/or selected subset of reference patient data sets (e.g., using statistics, machine learning, neural networks, AI, or the like). The predictive model can be configured to generate the surgical procedure and/or medical device design. The predictive model can determine access path(s) for surgically accessing targeted tissue. For example, the predictive model can identify suitable access paths between or around the vertebrae (e.g., vertebrae 52, 54 of FIGS. 1-2C) for accessing target sites associated with nerve compression. The system can analyze data sets to identify targeted tissue that should be removed to limit, minimize, or substantially prevent nerve compression associated with a level. The system can then design instruments for accessing the targeted tissue based on the access paths. Simulations can be repeatedly performed to model decompression steps. For example, patient-specific instruments can be configured to access the targeted tissue via the access paths to perform one or more decompression steps. A series of decompression steps can be performed at different sites along the spine to achieve overall reduction of nerve compression.

In some embodiments, the predictive model includes one or more trained machine learning models that generate, at least partly, the surgical procedure and/or medical device design. For example, the trained machine learning model(s) can determine a plurality of candidate surgical procedures and/or medical device designs for treating the patient. Each surgical procedure can be associated with one or more target sites (e.g., site for removal to provide an access path, decompression site, etc.), implantation sites, and/or corresponding medical device designs. In some embodiments, the surgical procedures and/or medical device designs are determined based on surgical procedure data and/or medical device design data associated with favorable outcomes, as previously described with respect to the data analysis phase 510. For each surgical procedure and/or corresponding medical device design, the trained machine learning model(s) can calculate a probability of achieving a target outcome (e.g., favorable or desired outcome) for the patient. The trained machine learning model(s) can then select at least one surgical procedure and/or corresponding medical device design based, at least partly, on the calculated probabilities.

The execution phase 530 can include manufacturing the medical device design (block 532). In some embodiments, the medical device design is manufactured by a manufacturing system configured to perform one or more of additive manufacturing, 3D printing, stereolithography, digital light processing, fused deposition modeling, selective laser sintering, selective laser melting, electronic beam melting, laminated object manufacturing, powder bed printing, thermoplastic printing, direct material deposition, or inkjet photo resin printing. The execution phase 530 can optionally include generating fabrication instructions configured to cause the manufacturing system to manufacture a medical device having the medical device design.

The execution phase 530 can include performing the surgical procedure (block 534). The surgical procedure can involve implanting a medical device having the medical device design into the patient. The surgical procedure can be performed manually, by a surgical robot, or a combination thereof. In embodiments where the surgical procedure is performed by a surgical robot, the execution phase 530 can include generating control instructions configured to cause the surgical robot to perform, at least partly, the patient-specific surgical procedure.

The method 500 can be implemented and performed in various ways. In some embodiments, one or more steps of the method 500 (e.g., the data phase 510 and/or the modeling phase 520) can be implemented as computer-readable instructions stored in memory and executable by one or more processors of any of the computing devices and systems described herein (e.g., the system 300), or a component thereof (e.g., the client computing device 302 and/or the server 306). Alternatively, one or more steps of the method 500 (e.g., the execution phase 530) can be performed by a healthcare provider (e.g., physician, surgeon), a robotic apparatus (e.g., a surgical robot), a manufacturing system (e.g., manufacturing system 324), or a combination thereof. In some embodiments, one or more steps of the method 500 are omitted (e.g., the execution phase 530).

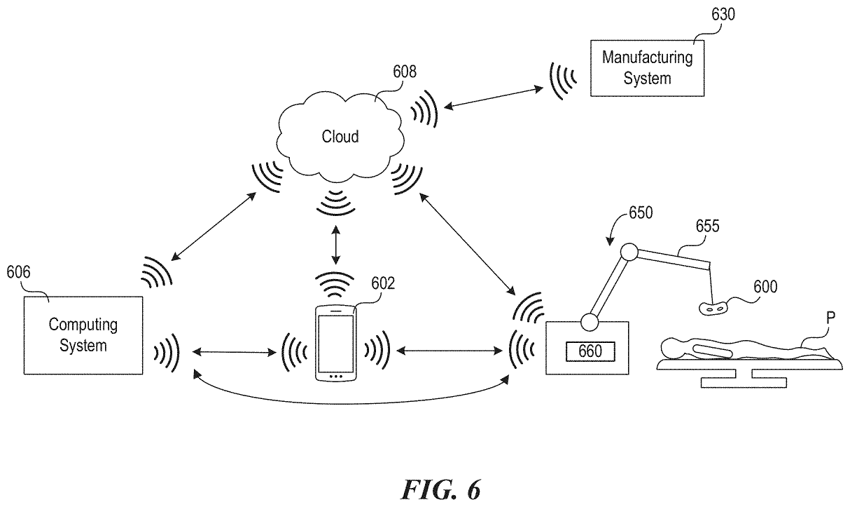

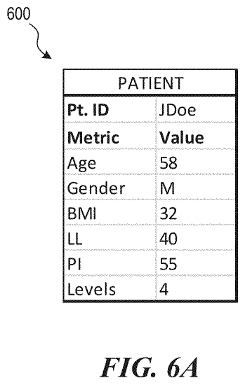

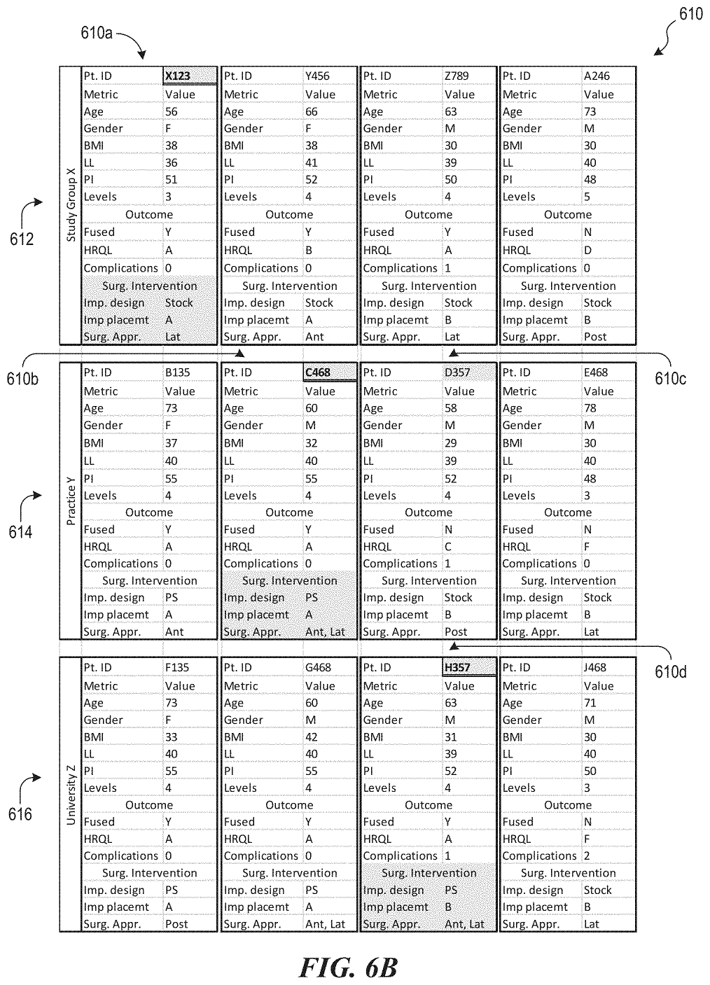

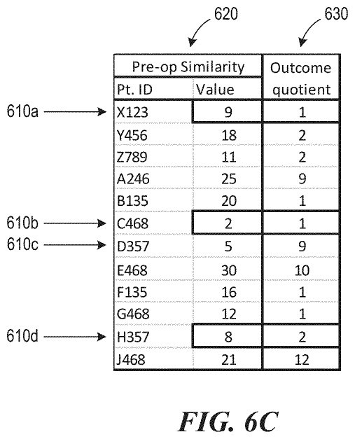

FIG. 6 is a schematic illustration of an operative setup including select systems and devices that can be used to provide patient-specific medical care, such as for performing the method 500 described with respect to FIG. 5. As shown, the operative setup includes a computing device 602, a computing system 606, a cloud 608, a manufacturing system 630, and a robotic surgical platform 650. The computing device 602 can be a user device, such as a smart phone, mobile device, laptop, desktop, personal computer, tablet, phablet, or other such devices known in the art. In operation, a user (e.g., a surgeon) can collect, retrieve, review, modify, or otherwise interact with a patient data set using the computing device 602. The computing system 606 can include any suitable computing system configured to store one or more software modules for identifying reference patient data sets, determining patient-specific surgical plans, generating virtual models of patient anatomy, designing patient-specific implants, or the like. The one or more software modules can include algorithms, machine-learning models, artificial intelligence architectures, or the like for performing select operations. The cloud 608 can be any suitable network and/or storage system, and may include any combination of hardware and/or virtual computing resources. The manufacturing system 630 can be any suitable manufacturing system for producing patient-specific implants, including any of those previously described herein. The robotic surgical platform 650 (referred to herein as “the platform 650”) can be configured to perform or otherwise assist with one or more aspects of a surgical procedure.

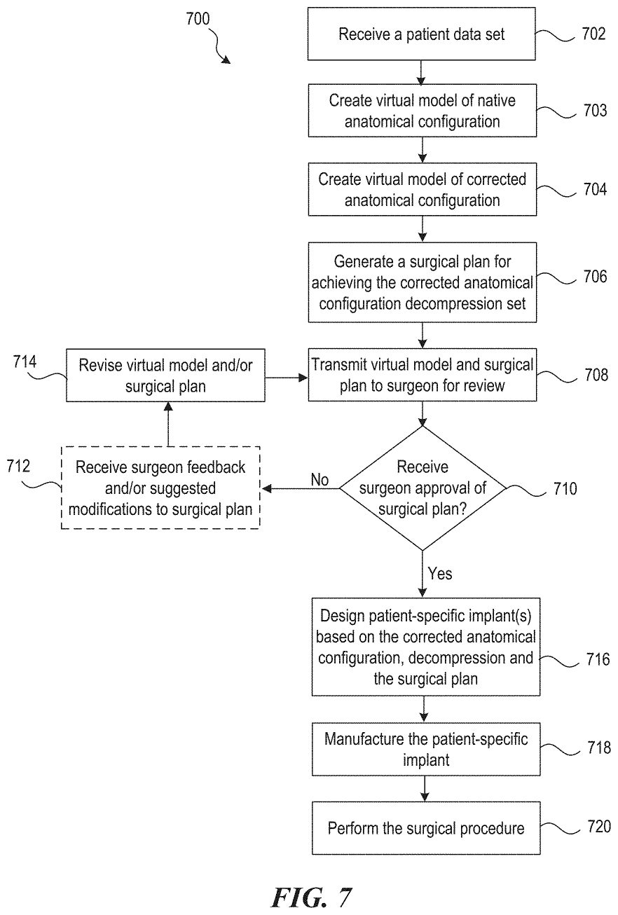

In a representative operation, the computing device 602, the computing system 606, the cloud 608, the manufacturing system 630, and the platform 650 can be used to provide patient-specific medical care, such as to perform the method 700 described with respect to FIG. 7. For example, the computing system 606 can receive a patient data set from the computing device 602 (e.g., step 702 of the method 700). In some embodiments, the computing device 602 can directly transmit the patient data set to the computing system 606. In other embodiments, the computing device 602 can upload the patient data set into the cloud 608, and the computing system 606 can download or otherwise access the patient data set from the cloud. Once the computing system 606 receives the patient data set, the computing system 606 can create a virtual model of the patient's native anatomical configuration (e.g., step 703 of the method 700), create a virtual model of the corrected anatomical configuration (e.g., step 704 of the method 700), and/or generate a surgical plan for achieving the corrected anatomical configuration (e.g., step 706 of the method 700). The computing system can perform the foregoing operations via the one or more software modules, which in some embodiments include machine learning models or other artificial intelligence architectures. Once the virtual models and the surgical plan are created, the computing system 606 can transmit the virtual models and the surgical plan to the surgeon for review (e.g., step 708 of the method 700). This can include, for example, directly transmitting the virtual models and the surgical plan to the computing device 602 for surgeon review. In other embodiments, this can include uploading the virtual models and the surgical plan to the cloud 608. A surgeon can then download or otherwise access the virtual models and the surgical plan from the cloud 608 using the computing device 602.

The surgeon can use the computing device 602 to review the virtual models and the surgical plan. The surgeon can also approve or reject the surgical plan and provide any feedback regarding the surgical plan using the computing device 602. The surgeon's approval, rejection, and/or feedback regarding the surgical plan can be transmitted to, and received by, the computing system 606 (e.g., steps 710 and 712 of the method 700). The computing system 606 can than revise the virtual model and/or the surgical plan (e.g., step 714 of the method 700). The computing system 606 can transmit the revised virtual model and surgical plan to the surgeon for review (e.g., by uploading it to the cloud 608 or directly transmitting it to the computing device 602).

The computing system 606 can also design the patient-specific implant based on the corrected anatomical configuration and the surgical plan (e.g., step 716 of the method 700) using, the one or more software modules. In some embodiments, software modules rely on one or more algorithms, machine learning models, or other artificial intelligence architectures to design the implant. Once the computing system 606 designs the patient-specific implant, the computing system 606 can upload the design and/or manufacturing instructions to the cloud 608. The computing system 606 may also create fabrication instructions (e.g., computer-readable fabrication instructions) for manufacturing the patient-specific implant. In such embodiments, the computing system 606 can upload the fabrication instructions to the cloud 608.

The manufacturing system 630 can download or otherwise access the design and/or fabrication instructions for the patient-specific implant from the cloud 608. The manufacturing system can then manufacture the patient-specific implant (e.g., step 718 in the method 700) using additive manufacturing techniques, subtractive manufacturing techniques, or other suitable manufacturing techniques.

The robotic surgical platform 650 can perform or otherwise assist with one or more aspects of the surgical procedure (e.g., step 720 of the method 700). For example, the platform 650 can prepare tissue for an incision, make an incision, make a resection, remove tissue, manipulate tissue, perform a corrective maneuver, deliver the implant to a target site, deploy the implant at the target site, adjust the implant at the target site, manipulate the implant once it is implanted, secure the implant at the target site, explant the implant, suture tissue, etc. The platform 650 can therefore include one or more arms 655 and end effectors for holding various surgical tools (e.g., graspers, clips, needles, needle drivers, irrigation tools, suction tools, staplers, screw driver assemblies, etc.), imaging instruments (e.g., cameras, sensors, etc.), and/or medical devices (e.g., the implant 600) and that enable the platform 650 to perform the one or more aspects of the surgical plan. Although shown as having one arm 655, one skilled in the art will appreciate that the platform 650 can have a plurality of arms (e.g., two, three, four, or more) and any number of joints, linkages, motors, and degrees of freedom. In some embodiments, the platform 650 may have a first arm dedicated to holding one or more imaging instruments, while the remainder of the arms hold various surgical tools. In some embodiments, the tools can be releasably secured to the arms such that they can be selectively interchanged before, during, or after an operative procedure. The arms can be moveable through a variety of ranges of motion (e.g., degrees of freedom) to provide adequate dexterity for performing various aspects of the operative procedure.

The platform 650 can include a control module 660 for controlling operation of the arm(s) 655. In some embodiments, the control module 660 includes a user input device (not shown) for controlling operation of the arm(s) 655. The user input device can be a joystick, a mouse, a keyboard, a touchscreen, an infrared sensor, a touchpad, a wearable input device, a camera- or image-based input device, a microphone, or other user input devices. A user (e.g., a surgeon) can interact with the user input device to control movement of the arm(s) 655.

In some embodiments, the control module 660 includes one or more processors for executing machine-readable operative instructions that, when executed, automatically control operation of the arm 655 to perform one or more aspects of the surgical procedure. In some embodiments, the control module 660 may receive the machine-readable operative instructions (e.g., from the cloud 608) specifying one or more steps of the surgical procedure that, when executed by the control module 660, cause the platform 650 to perform the one or more steps of the surgical procedure. For example, the machine-readable operative instructions may direct the platform 650 to prepare tissue for an incision, make an incision, make a resection, remove tissue, manipulate tissue, perform a corrective maneuver, deliver the implant 600 to a target site, deploy the implant 600 at the target site, adjust a configuration of the implant 600 at the target site, manipulate the implant 600 once it is implanted, secure the implant 600 at the target site, explant the implant 600, suture tissue, and the like. The operative instructions may therefor include particular instructions for articulating the arm 655 to perform or otherwise aid in the delivery of the patient-specific implant.

In some embodiments, the platform 650 can generate (e.g., as opposed to simply receiving) the machine-readable operative instructions based on the surgical plan. For example, the surgical plan can include information about the delivery path, tools, and implantation site. The platform 650 can analyze the surgical plan and develop executable operative instructions for performing the patient-specific procedure based on the capabilities (e.g., configuration and number of robotic arms, functionality of and effectors, guidance systems, visualization systems, etc.) of the robotic system. This enables the operative setup shown in FIG. 6 to be compatible with a wide range of different types of robotic surgery systems.

The platform 650 can include one or more communication devices (e.g., components having VLC, WiMAX, LTE, WLAN, IR communication, PSTN, Radio waves, Bluetooth, and/or Wi-Fi operability) for establishing a connection with the cloud 608 and/or the computing device 602 for accessing and/or downloading the surgical plan and/or the machine-readable operative instructions. For example, the cloud 608 can receive a request for a particular surgical plan from the platform 650 and send the plan to the platform 650. Once identified, the cloud 608 can transmit the surgical plan directly to the platform 650 for execution. In some embodiments, the cloud 608 can transmit the surgical plan to one or more intermediate networked devices (e.g., the computing device 602) rather than transmitting the surgical plan directly to the platform 650. A user can review the surgical plan using the computing device 602 before transmitting the surgical plan to the platform 650 for execution. Additional details for identifying, storing, downloading, and accessing patient-specific surgical plans are described in U.S. application Ser. No. 16/990,810, filed Aug. 11, 2020, the disclosure of which is incorporated by reference herein in its entirety.

The platform 650 can include additional components not expressly shown in FIG. 6. For example, in various embodiments the platform 650 may include one or more displays (e.g., LCD display screen, an LED display screen, a projected, holographic, or augmented reality display (e.g., a heads-up display device or a head-mounted device), one or more I/O devices (e.g., a network card, video card, audio card, USB, firewire or other external device, camera, printer, speakers, CD-ROM drive, DVD drive, disk drive, or Blu-Ray device), and/or a memory (e.g., random access memory (RAM), various caches, CPU registers, read-only memory (ROM), and writable non-volatile memory, such as flash memory, hard drives, floppy disks, CDs, DVDs, magnetic storage devices, tape drives, device buffers, and so forth). In some embodiments, the foregoing components can be generally similar to the like components described in detail with respect to computing device 400 in FIG. 4.

Without being bound by theory, using a robotic surgical platform to perform various aspects of the surgical plans described herein is expected to provide several advantages over conventional operative techniques. For example, use of robotic surgical platforms may improve surgical outcomes and/or shorten recovery times by, for example, decreasing incision size, decreasing blood loss, decreasing a length of time of the operative procedure, increasing the accuracy and precision of the surgery (e.g., the placement of the implant at the target location), and the like. The platform 650 can also avoid or reduce user input errors, e.g., by including one or more scanners for obtaining information from instruments (e.g., instruments with retrieval features), tools, the patient-specific implant 600 (e.g., after the implant 600 has been gripped by the arm 655), etc. The platform 650 can confirm use of proper instruments prior and during the surgical procedure. If the platform 650 identifies an incorrect instrument or tool, an alert can be sent to a user that another instrument or tool should be installed. The user can scan the new instrument to confirm that the instrument is appropriate for the surgical plan. In some embodiments, the surgical plan includes instructions for use, a list of instruments, instrument specifications, replacement instruments, and the like. The platform 650 can perform pre- and post-surgical checking routines based on information from the scanners.

FIGS. 6A-6C illustrate exemplary data sets that may be used and/or generated in connection with the methods described herein (e.g., the data analysis phase 510 described with respect to FIG. 5 and system of FIG. 6), according to an embodiment. FIG. 6A illustrates a patient data set 601 of a patient to be treated. The patient data set 600 can include a patient ID and a plurality of pre-operative patient metrics (e.g., age, gender, BMI, lumbar lordosis (LL), pelvic incidence (PI), and treatment levels of the spine (levels)). FIG. 6B illustrates a plurality of reference patient data sets 610. In the depicted embodiment, the reference patient data sets 610 include a first subset 612 from a study group (Study Group X), a second subset 614 from a practice database (Practice Y), and a third subset 616 from an academic group (University Z). In alternative embodiments, the reference patient data sets 610 can include data from other sources, as previously described herein. Each reference patient data set can include a patient ID, a plurality of pre-operative patient metrics (e.g., age, gender, BMI, lumbar lordosis (LL), pelvic incidence (PI), and treatment levels of the spine (levels), pain scores, pain metrics, nerve compression test scores, mobility scores, etc.), treatment outcome data (Outcome) (e.g., presence of fusion (fused), HRQL, complications), and treatment procedure data (Surg. Intervention) (e.g., implant design, implant placement, surgical approach).

FIG. 6C illustrates comparison of the patient data set 601 to the reference patient data sets 610. As previously described, the patient data set 601 can be compared to the reference patient data sets 610 to identify one or more similar patient data sets from the reference patient data sets. In some embodiments, the patient metrics from the reference patient data sets 610 are converted to numeric values and compared the patient metrics from the patient data set 601 to calculate a similarity score 620 (“Pre-op Similarity”) for each reference patient data set. Reference patient data sets having a similarity score below a threshold value can be considered to be similar to the patient data set 601. For example, in the depicted embodiment, reference patient data set 610a has a similarity score of 9, reference patient data set 610b has a similarity score of 2, reference patient data set 610c has a similarity score of 5, and reference patient data set 610d has a similarity score of 8. Because each of these scores are below the threshold value of 10, reference patient data sets 610a-d are identified as being similar patient data sets.

The treatment outcome data of the similar patient data sets 610a-d can be analyzed to determine surgical procedures and/or implant designs with the highest probabilities of success. For example, the treatment outcome data for each reference patient data set can be converted to a numerical outcome score 630 (“Outcome Quotient”) representing the likelihood of a favorable outcome. In the depicted embodiment, reference patient data set 610a has an outcome score of 1, reference patient data set 610b has an outcome score of 1, reference patient data set 610c has an outcome score of 9, and reference patient data set 610d has an outcome score of 2. In embodiments where a lower outcome score correlates to a higher likelihood of a favorable outcome, reference patient data sets 610a, 610b, and 610d can be selected. The treatment procedure data from the selected reference patient data sets 610a, 610b, and 610d can then be used to determine at least one surgical procedure (e.g., implant placement, surgical approach) and/or implant design that is likely to produce a favorable outcome for the patient to be treated.

In some embodiments, a method for providing medical care to a patient is provided. The method can include comparing a patient data set to reference data. The patient data set and reference data can include any of the data types described herein. The method can include identifying and/or selecting relevant reference data (e.g., data relevant to treatment of the patient, such as data of similar patients and/or data of similar treatment procedures), using any of the techniques described herein. A treatment plan can be generated based on the selected data, using any of the techniques described herein. The treatment plan can include one or more treatment procedures (e.g., surgical procedures, instructions for procedures, models or other virtual representations of procedures), one or more medical devices (e.g., implanted devices, instruments for delivering devices, surgical kits), or a combination thereof.

In some embodiments, a system for generating a medical treatment plan is provided. The system can compare a patient data set to a plurality of reference patient data sets, using any of the techniques described herein. A subset of the plurality of reference patient data sets can be selected, e.g., based on similarity and/or treatment outcome, or any other technique as described herein. A medical treatment plan can be generated based at least in part on the selected subset, using any of the techniques described herein. The medical treatment plan can include one or more treatment procedures, one or more medical devices, or any of the other aspects of a treatment plan described herein, or combinations thereof.

In further embodiments, a system is configured to use historical patient data. The system can select historical patient data to develop or select a treatment plan, design medical devices, or the like. Historical data can be selected based on one or more similarities between the present patient and prior patients to develop a prescriptive treatment plan designed for desired outcomes. The prescriptive treatment plan can be tailored for the present patient to increase the likelihood of the desired outcome. In some embodiments, the system can analyze and/or select a subset of historical data to generate one or more treatment procedures, one or more medical devices, or a combination thereof. In some embodiments, the system can use subsets of data from one or more groups of prior patients, with favorable outcomes, to produce a reference historical data set used to, for example, design, develop or select the treatment plan, medical devices, or combinations thereof.

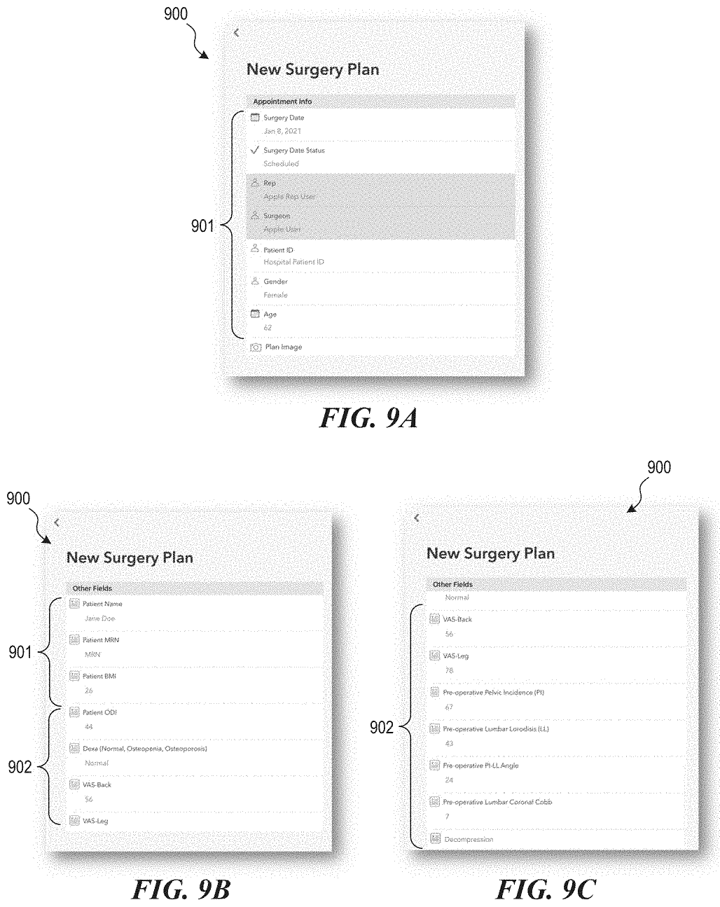

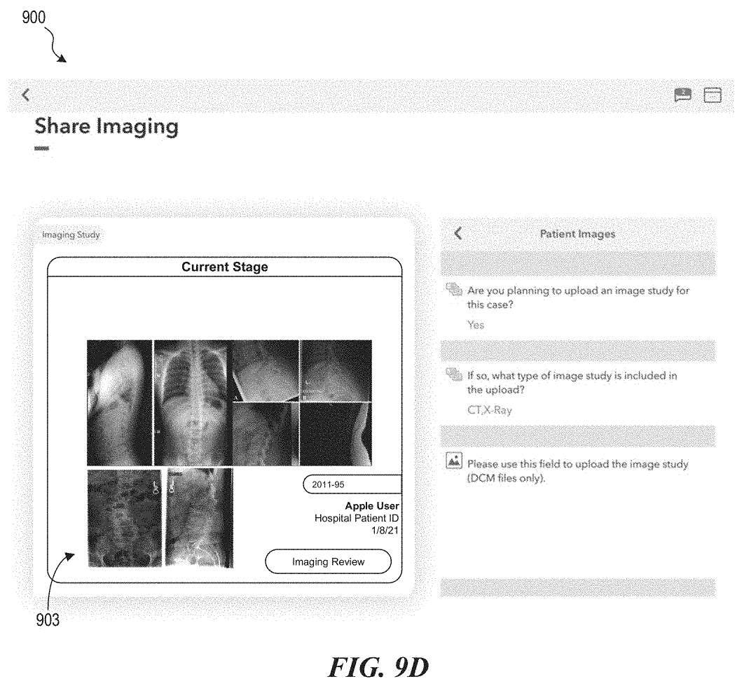

FIG. 7 is a flow diagram illustrating a method 700 for providing patient-specific medical care, according to another embodiment of the present technology. The method 700 can begin in step 702 by receiving a patient data set for a particular patient in need of medical treatment. The patient data set can include data representative of the patient's condition, anatomy, pathology, symptoms, medical history, preferences, and/or any other information or parameters relevant to the patient. For example, the patient data set 808 can include surgical intervention data, treatment outcome data, progress data (e.g., surgeon notes), patient feedback (e.g., feedback acquired using quality of life questionnaires, surveys), clinical data, patient information (e.g., demographics, sex, age, height, weight, type of pathology, occupation, activity level, tissue information, health rating, comorbidities, health related quality of life (HRQL)), vital signs, diagnostic results, medication information, allergies, diagnostic equipment information (e.g., manufacturer, model number, specifications, user-selected settings/configurations, etc.) or the like. The patient data set can also include image data, such as camera images, Magnetic Resonance Imaging (MRI) images, ultrasound images, Computerized Aided Tomography (CAT) scan images, Positron Emission Tomography (PET) images, X-Ray images, and the like. In some embodiments, the patient data set includes data representing one or more of patient identification number (ID), age, gender, body mass index (BMI), lumbar lordosis, Cobb angle(s), pelvic incidence, disc height, segment flexibility, bone quality, rotational displacement, and/or treatment level of the spine. The patient data set can be received at a server, computing device, or other computing system. For example, in some embodiments the patient data set can be received by the server 306 shown in FIG. 3 or the computing system 606 described above with respect to FIG. 6. In some embodiments, the computing system that receives the patient data set in step 702 also stores one or more software modules (e.g., the data analysis module 316 and/or the treatment planning module 318, shown in FIG. 3, or additional software modules for performing various operations of the method 700). Additional details for collecting and receiving the patient data set are described below with respect to FIGS. 9A-9D.

In some embodiments, the received patient data set can include disease metrics such as lumbar lordosis, Cobb angles, coronal parameters (e.g., coronal balance, global coronal balance, coronal pelvic tilt, etc.), sagittal parameters (e.g., pelvic incidence, sacral slope, thoracic kyphosis, etc.) and/or pelvic parameters. The disease metrics can include micro-measurements (e.g., metrics associated with specific or individual segments of the patient's spine) and/or macro-measurements (e.g., metrics associated with multiple segments of the patient's spine). In some embodiments, the disease metrics are not included in the patient data set, and the method 700 includes determining (e.g., automatically determining) one or more of the disease metrics based on the patient image data, as described below.

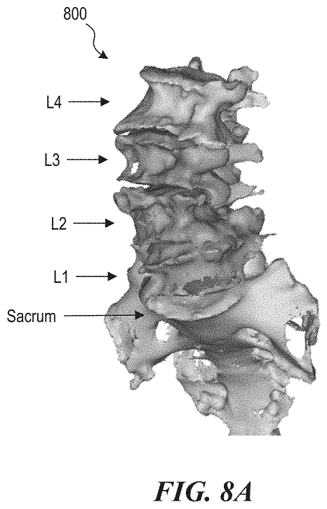

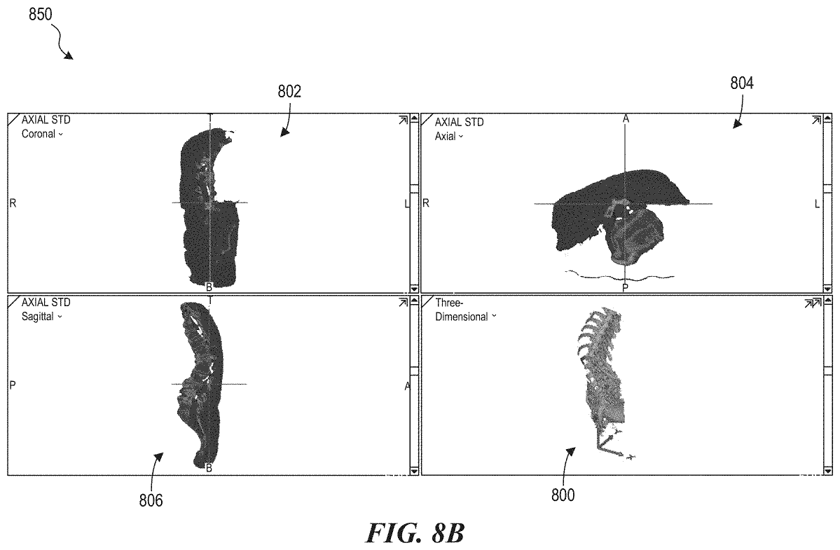

Once the patient data set is received in step 702, the method 700 can continue in step 703 by creating a virtual model of the patient's native anatomical configuration (also referred to as “pre-operative anatomical configuration”). The virtual model can be based on the image data included in the patient data set received in step 702. For example, the same computing system that received the patient data set in step 702 can analyze the image data in the patient data set to generate a virtual model of the patient's native anatomical configuration. The virtual model can be a two- or three-dimensional visual representation of the patient's native anatomy. The virtual model can include one or more regions of interest, and may include some or all of the patient's anatomy within the regions of interest (e.g., any combination of tissue types including, but not limited to, bony structures, cartilage, soft tissue, vascular tissue, nervous tissue, etc.). As a non-limiting example, the virtual model can include a visual representation of the patient's spinal cord region, including some or all of the sacrum, lumbar region, thoracic region, and/or cervical region. In some embodiments, the virtual model includes soft tissue, cartilage, and other non-bony structures. In other embodiments, the virtual model only includes the patient's bony structures. An example of a virtual model of the native anatomical configuration is described below with respect to FIGS. 8A and 8B. In some embodiments, the method 700 can optionally omit creating a virtual model of the patient's native anatomy in step 703, and proceed directly from step 702 to step 704.

In some embodiments, the computing system that generated the virtual model in step 702 can also determine (e.g., automatically determine or measure) one or more disease metrics of the patient based on the virtual model. For example, the computing system may analyze the virtual model to determine the patient's pre-operative lumbar lordosis, Cobb angles, coronal parameters (e.g., coronal balance, global coronal balance, coronal pelvic tilt, etc.), sagittal parameters (e.g., pelvic incidence, sacral slope, thoracic kyphosis, etc.) and/or pelvic parameters. The disease metrics can include micro-measurements (e.g., metrics associated with specific or individual segments of the patient's spine) and/or macro-measurements (e.g., metrics associated with multiple segments of the patient's spine).

The method 700 can continue in step 704 by creating a virtual model of a corrected anatomical configuration (which can also be referred to herein as the “planned configuration,” “optimized geometry,” “post-operative anatomical configuration,” or “target outcome”) for the patient. For example, the computing system can, using the analysis procedures described previously, determine a “corrected” or “optimized” anatomical configuration for the particular patient that represents an ideal surgical outcome for the particular patient. This can be done, for example, by analyzing a plurality of reference patient data sets to identify post-operative anatomical configurations for similar patients who had a favorable post-operative outcome, as previously described in detail with respect to FIGS. 3-6C (e.g., based on similarity of the reference patient data set to the patient data set and/or whether the reference patient had a favorable treatment outcome). This may also include applying one or more mathematical rules defining optimal anatomical outcomes (e.g., positional relationships between anatomic elements) and/or target (e.g., acceptable) post-operative metrics/design criteria (e.g., adjust anatomy so that the post-operative sagittal vertical axis is less than 7 mm, the post-operative Cobb angle less than 10 degrees, etc.). Target post-operative metrics can include, but are not limited to, target coronal parameters, target sagittal parameters, target pelvic incidence angle, target Cobb angle, target shoulder tilt, target iliolumbar angle, target coronal balance, target Cobb angle, target lordosis angle, and/or a target intervertebral space height. The different between the native anatomical configuration and the corrected anatomical configuration may be referred to as a “patient-specific correction” or “target correction.”

Once the corrected anatomical configuration is determined, the computing system can generate a two- or three-dimensional visual representation of the patient's anatomy with the corrected anatomical configuration. As with the virtual model created in step 703, the virtual model of the patient's corrected anatomical configuration can include one or more regions of interest, and may include some or all of the patient's anatomy within the regions of interest (e.g., any combination of tissue types including, but not limited to, bony structures, cartilage, soft tissue, vascular tissue, nervous tissue, etc.). As a non-limiting example, the virtual model can include a visual representation of the patient's spinal cord region in a corrected anatomical configuration, including some or all of the sacrum, lumbar region, thoracic region, and/or cervical region. In some embodiments, the virtual model includes soft tissue, cartilage, and other non-bony structures. In other embodiments, the virtual model only includes the patient's bony structures. An example of a virtual model of the native anatomical configuration is described below with respect to FIGS. 10A-1-10B-2.

Simulations can be used to evaluate the decompression of tissue. In some embodiments, a simulation can include simulating one or more spinal corrections by moving features of a three-dimensional virtual model of a patient's anatomy. The simulation can be used to identify compression of tissue (e.g., nerve roots, nerve tissue, etc.) caused by the movement of the features of the three-dimensional virtual model. A surgical plan, instruments, and other items disclosed herein can be modified to reduce the identified compression of the tissue. Each surgical step can be simulated to enable the designing of items based on the predicted anatomy for that step. The predicted anatomy can include modified anatomical features, enlarged working spaces, etc. The models discussed in connection with, for example, FIGS. 10A-1-10B-2 and 11-13B dan be used to predict anatomical configurations, including intra-operative configurations, post-operative configuration, long-term configurations, etc.

The method 700 can continue in step 706 by generating (e.g., automatically generating) a surgical plan for achieving the corrected anatomical configuration, decompression, and/or other outcomes shown by the virtual model. The corrected anatomical configuration may increase or decrease nerve compression at various levels along the spine. The system can identify changes in tissue compression associated with the corrected anatomical configuration and can develop ancillary steps to compensate for such compression. In some procedures, anatomical corrections may cause or contribute to nerve compression. The system can identify the predicted nerve compression and develop a decompression plan for reducing or eliminating such predicted nerve compression. The decompression plan can be incorporated into a comprehensive surgical plan for correcting vertebral positioning while also limiting or preventing unwanted side effects.

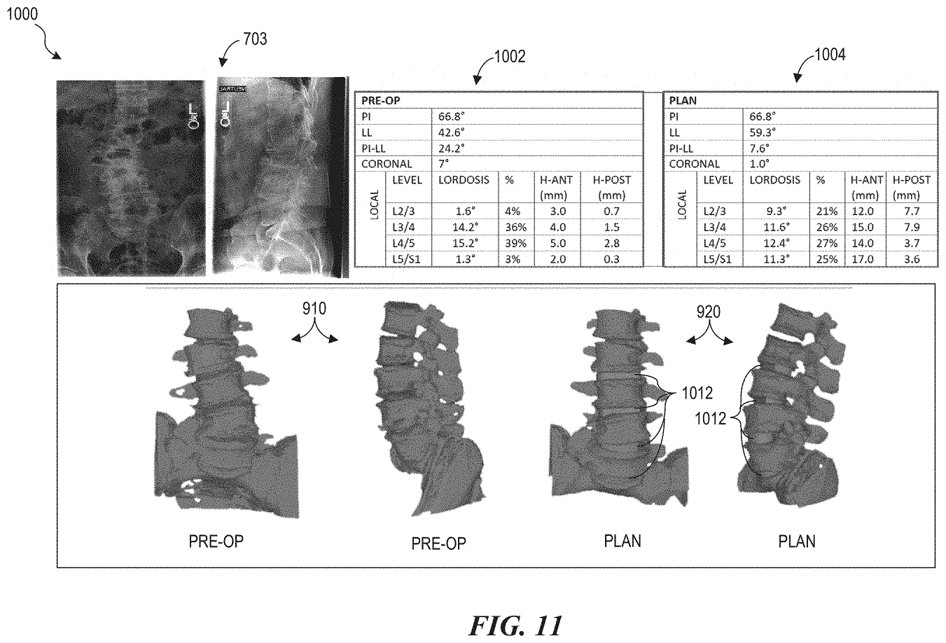

The surgical plan can include pre-operative plans, operative plans, post-operative plans, and/or specific spine metrics associated with the optimal surgical outcome. For example, the surgical plans can include a specific surgical procedure for achieving the corrected anatomical configuration. In the context of spinal surgery, the surgical plan may include a specific fusion surgery (e.g., PLIF, ALIF, TLIF, LLIF, DLIF, XLIF, etc.) across a specific range of vertebral levels (e.g., L1-L4, L1-5, L3-T12, etc.). Of course, other surgical procedures may be identified for achieving the corrected anatomical configuration, such as non-fusion surgical approaches and orthopedic procedures for other areas of the patient. The surgical plan may also include one or more expected spine metrics (e.g., lumbar lordosis, Cobb angles, coronal parameters, sagittal parameters, and/or pelvic parameters) corresponding to the expected post-operative patient anatomy. The surgical plan can be generated by the same or different computing system that created the virtual model of the corrected anatomical configuration. In some embodiments, the surgical plan can also be based on one or more reference patient data sets as previously described with respect to FIGS. 3-6C. In some embodiments, the surgical plan can also be based at least in part on surgeon-specific preferences and/or outcomes associated with a specific surgeon performing the surgery. In some embodiments, more than one surgical plan is generated in step 706 to provide a surgeon with multiple options. An example of a surgical plan is described below with respect to FIG. 11.

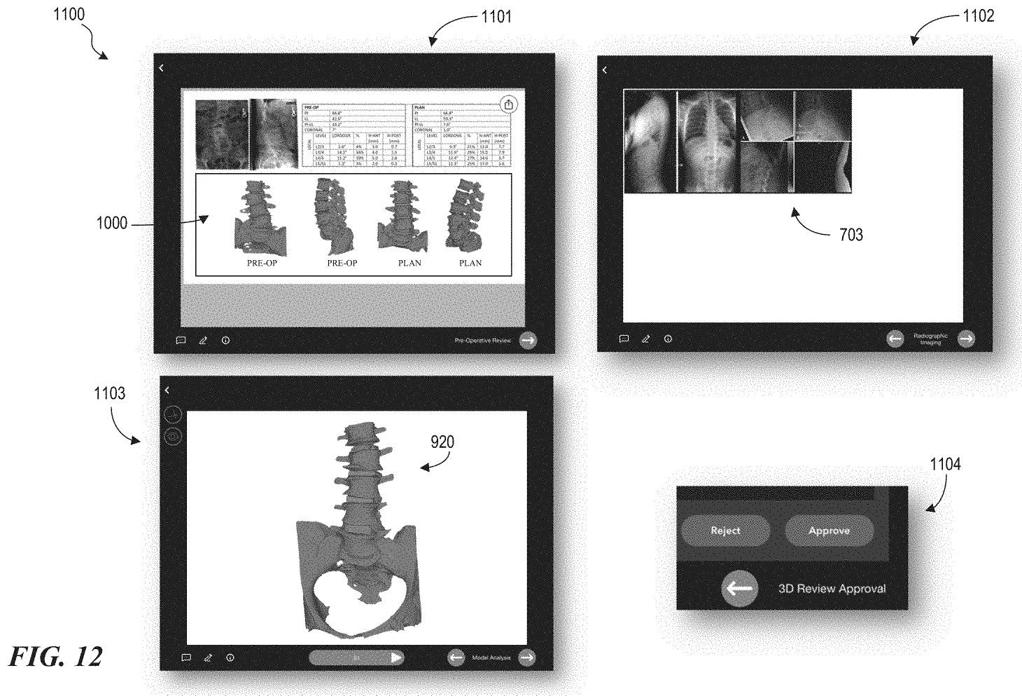

After the virtual model of the corrected anatomical configuration is created in step 704 and the surgical plan is generated in step 706, the method 700 can continue in step 708 by transmitting the virtual model of the corrected anatomical configuration and the surgical plan for surgeon review. In some embodiments, the virtual model and the surgical plan are transmitted as a surgical plan report, an example of which is described with respect to FIG. 12. In some embodiments, the same computing system used in steps 702-706 can transmit the virtual model and surgical plan to a computing device for surgeon review (e.g., the client computing device 302 described in FIG. 3 or the computing device 602 described above with respect to FIG. 6). This can include directly transmitting the virtual model and the surgical plan to the computing device or uploading the virtual model and the surgical plan to a cloud or other storage system for subsequent downloading. Although step 708 describes transmitting the surgical plan and the virtual model to the surgeon, one skilled in the art will appreciate from the disclosure herein that images of the virtual model may be included in the surgical plan transmitted to the surgeon, and that the actual model need not be included (e.g., to decrease the file size being transmitted). Additionally, the information transmitted to the surgeon in step 708 may include the virtual model of the patient's native anatomical configuration (or images thereof) in addition to the virtual model of the corrected anatomical configuration. In embodiments in which more than one surgical plan is generated in step 706, the method 700 can include transmitting more than one surgical plan to the surgeon for review and selection.

The surgeon can review the virtual model and surgical plan and, in step 710, either approve or reject the surgical plan (or, if more than one surgical plan is provided in step 708, select one of the provided surgical plans). If the surgeon does not approve the surgical plan in step 710, the surgeon can optionally provide feedback and/or suggested modifications to the surgical plan (e.g., by adjusting the virtual model or changing one or more aspects about the plan). Accordingly, the method 700 can include receiving (e.g., via the computing system) the surgeon feedback and/or suggested modifications. If surgeon feedback and/or suggested modifications are received in step 712, the method 700 can continue in step 714 by revising (e.g., automatically revising via the computing system) the virtual model and/or surgical plan based at least in part on the surgeon feedback and/or suggested modifications received in step 712. In some embodiments, the surgeon does not provide feedback and/or suggested modifications if they reject the surgical plan. In such embodiments, step 712 can be omitted, and the method 700 can continue in step 714 by revising (e.g., automatically revising via the computing system) the virtual model and/or the surgical plan by selecting new and/or additional reference patient data sets. The revised virtual model and/or surgical plan can then be transmitted to the surgeon for review. Steps 708, 710, 712, and 714 can be repeated as many times as necessary until the surgeon approves the surgical plan. Although described as the surgeon reviewing, modifying, approving, and/or rejecting the surgical plan, in some embodiments the surgeon can also review, modify, approve, and/or reject the corrected anatomical configuration shown via the virtual model.

In some embodiments, step 714 includes revising the virtual model and/or surgical plan based on predicted nerve compression. The system can analyze spinal corrections to identify pre-operative, intra-operative, and/or post-operative nerve compression. The system can then predict post-operative nerve compression scores to determine whether they exceed a threshold level. If the nerve compression scores exceed the threshold level, the system can determine whether to modify or revise the surgical plan to reduce the predicted nerve compression scores to be below the threshold level or whether to generate a decompression plan and patient-specific instruments to perform decompression procedures to reduce or limit post-operative nerve compression. In some embodiments, at steps 710 and/or 712, the surgeon can review options for predicting, scoring, selecting threshold levels, and/or treating predicted nerve compression. In some embodiments, the surgeon can approve identified targeted tissue that can be removed from the patient to achieve the elimination or targeted reduction of nerve compression.

Once surgeon approval of the surgical plan is received in step 710, the method 700 can continue in step 716 by designing (e.g., via the same computing system that performed steps 702-714 of FIG. 7) a patient-specific instruments, patient-specific implant, and other technology based on the corrected anatomical configuration and the surgical plan. The patient-specific instruments can be designed for the pre-corrected anatomical configuration, post-corrected anatomical configuration, or both. In some procedures, the decompression steps can be performed prior to correcting the anatomical configuration of the patient because the uncorrected anatomical configuration may provide more access to nerve compression sites. In other procedures, the corrected anatomical configuration may facilitate access to compression sites by increasing spacing for enlarged access paths. In such scenarios, decompression instruments can be designed to perform one or more decompression steps after anatomical configuration correction (e.g., spine realignment, restoration discs heights, etc.). In some procedures, decompression steps can be performed before, during, and/or after correcting the anatomical configuration to access compression sites at optimal times.

The system can design decompression and fusion operations in tandem to provide synergistic outcomes. For example, the system can consider both decompression and fusion operations (including synergies) when designing a surgical plan, tools, etc. to achieve improved outcomes as compared to designing decompression procedure and then designing a fusion procedure, or vice versa. In some embodiment, the systems sequentially design decompression and fusion procedures based on, for example, the priority of each procedure, user input, etc. In some embodiments, the system can design decompression plans, instruments, tools, etc. based on intraoperative mobility of the patient. U.S. App. No. 63/223,827 discloses intraoperative planning and predictions and is incorporated by reference in its entirety. The system can generate a virtual model of the patient's anatomy and can identifying one or more soft tissue surgical steps for adjusting intraoperative mobility of vertebrae of the spine to achieve an anatomical configuration for decompression operations, fusion operations, or other operations. The system can generate a plan that includes at least one of the soft tissue surgical steps that facilitates movement of the vertebrae to the corrected anatomical configuration and decompression steps.

The patient-specific implant can be specifically designed such that, when it is implanted in the particular patient, it directs the patient's anatomy to occupy the corrected anatomical configuration (e.g., transforming the patient's anatomy from the native anatomical configuration to the corrected anatomical configuration). The patient-specific implant can be designed such that, when implanted, it causes the patient's anatomy to occupy the corrected anatomical configuration for the expected service life of the implant (e.g., 5 years or more, 10 years or more, 20 years or more, 50 years or more, etc.). In some embodiments, the patient-specific implant is designed solely based on the virtual model of the corrected anatomical configuration and/or without reference to pre-operative patient images.

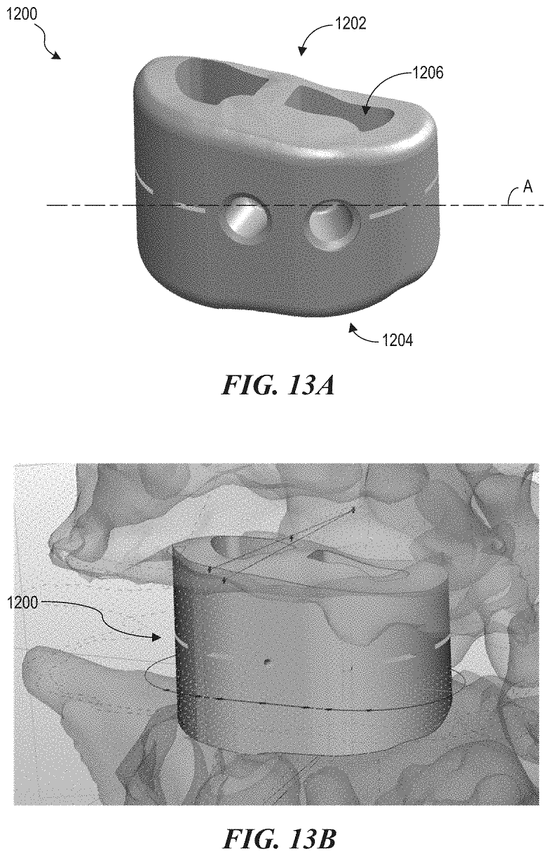

The patient-specific implant can be any of the implants described herein or in any patent references incorporated by reference herein. For example, the patient-specific implant can include one or more of screws (e.g., bone screws, spinal screws, pedicle screws, facet screws), interbody implant devices (e.g., intervertebral implants), cages, plates, rods, discs, fusion devices, spacers, rods, expandable devices, stents, brackets, ties, scaffolds, fixation device, anchors, nuts, bolts, rivets, connectors, tethers, fasteners, joint replacements (e.g., artificial discs), hip implants, or the like. A patient-specific implant design can include data representing one or more of physical properties (e.g., size, shape, volume, material, mass, weight), mechanical properties (e.g., stiffness, strength, modulus, hardness), and/or biological properties (e.g., osteo-integration, cellular adhesion, anti-bacterial properties, anti-viral properties) of the implant. For example, a design for an orthopedic implant can include implant shape, size, material, and/or effective stiffness (e.g., lattice density, number of struts, location of struts, etc.). An example of a patient-specific implant designed via the method 700 is described below with respect to FIGS. 13A and 13B.

In some embodiments, designing the implant in step 716 can optionally include generating fabrication instructions for manufacturing the implant. For example, the computing system may generate computer-executable fabrication instructions that that, when executed by a manufacturing system, cause the manufacturing system to manufacture the implant.

In some embodiments, the patient-specific implant is designed in step 716 only after the surgeon has reviewed and approved the virtual model with the corrected anatomical configuration and the surgical plan. Accordingly, in some embodiments, the implant design is neither transmitted to the surgeon with the surgical plan in step 708, nor manufactured before receiving surgeon approval of the surgical plan. Without being bound by theory, waiting to design the patient-specific implant until after the surgeon approves the surgical plan may increase the efficiency of the method 700 and/or reduce the resources necessary to perform the method 700.

The method 700 can continue in step 718 by manufacturing the patient-specific implant. The implant can be manufactured using additive manufacturing techniques, such as 3D printing, stereolithography, digital light processing, fused deposition modeling, selective laser sintering, selective laser melting, electronic beam melting, laminated object manufacturing, powder bed printing, thermoplastic printing, direct material deposition, or inkjet photo resin printing, or like technologies, or combination thereof. Alternatively or additionally, the implant can be manufactured using subtractive manufacturing techniques, such as CNC machining, electrical discharge machining (EDM), grinding, laser cutting, water jet machining, manual machining (e.g., milling, lathe/turning), or like technologies, or combinations thereof. The implant may be manufactured by any suitable manufacturing system (e.g., the manufacturing system 324 shown in FIG. 3 or the manufacturing system 630 described above with respect to FIG. 6). In some embodiments, the implant is manufactured by the manufacturing system executing the computer-readable fabrication instructions generated by the computing system in step 716.

Once the implant is manufactured in step 718, the method 700 can continue in step 720 by implanting the patient-specific implant into the patient. The surgical procedure can be performed manually, by a robotic surgical platform (e.g., a surgical robot), or a combination thereof. In embodiments in which the surgical procedure is performed at least in part by a robotic surgical platform, the surgical plan can include computer-readable control instructions configured to cause the surgical robot to perform, at least partly, the patient-specific surgical procedure. Additional details regarding a robotic surgical platform are described above with respect to FIG. 6.

The method 700 can be implemented and performed in various ways. In some embodiments, steps 702-716 can be performed by a computing system (e.g., the computing system 606 described above with respect to FIG. 6) associated with a first entity, step 718 can be performed by a manufacturing system associated with a second entity, and step 720 can be performed by a surgical provider, surgeon, and/or robotic surgical platform associated with a third entity. Any of the foregoing steps may also be implemented as computer-readable instructions stored in memory and executable by one or more processors of the associated computing system(s).

FIGS. 9A-14 further illustrate select aspects of providing patient-specific medical care, e.g., in accordance with the method 700. For example, FIGS. 9A-9D illustrate an example of a patient data set 900 (e.g., as received in step 702 of the method 700). The patient data set 900 can include any of the information previously described with respect to the patient data set. For example, the patient data set 900 includes patient information 901 (e.g., patient identification no., patient MRN, patient name, sex, age, body mass index (BMI), surgery date, surgeon, etc., shown in FIGS. 9A and 9B), diagnostic information 902 (e.g., Oswestry Disability Index (ODI), VAS-back score, VAS-leg score, pre-operative pelvic incidence, pre-operative lumbar lordosis, pre-operative PI-LL angle, pre-operative lumbar coronal Cobb, decompression information, etc., shown in FIGS. 9B and 9C), and image data 903 (x-ray, CT, MRI, etc., shown in FIG. 9D). In the illustrated embodiment, the patient data set 900 is collected by a healthcare provider (e.g., a surgeon, a nurse, etc.) using a digital and/or fillable report that can be accessed using a computing device (e.g., the computing device 602 shown in FIG. 6). In some embodiments, the patient data set 900 can be automatically or at least partially automatically generated based on digital medical records of the patient. Regardless, once collected, the patient data set 900 can be transmitted to the computing system configured to generate the surgical plan for the patient (e.g., the computing system 606 shown in FIG. 6).

FIGS. 8A and 8B illustrate an example of a virtual model 800 of a patient's native anatomical configuration (e.g., as created in step 703 of the method 700). In particular, FIG. 8A is an enlarged view of the virtual model 800 of the patient's native anatomy and shows the patient's native anatomy of their lower spinal cord region. The virtual model 800 is a three-dimensional visual representation of the patient's native anatomy. In the illustrated embodiment, the virtual model includes a portion of the spinal column extending from the sacrum to the L4 vertebral level. Of course, the virtual model can include other regions of the patient's spinal column, including cervical vertebrae, thoracic vertebrae, lumbar vertebrae, and the sacrum. The illustrated virtual model 800 only includes bony structures of the patient's anatomy, but in other embodiments may include additional structures, such as cartilage, soft tissue, vascular tissue, nervous tissue, etc.

FIG. 8B illustrates a virtual model display 850 (referred to herein as the “display 850”) showing different views of the virtual model 800. The virtual model display 850 includes a three-dimensional view of the virtual model 800, one or more coronal cross-section(s) 802 of the virtual model 800, one or more axial cross section(s) 804 of the virtual model 800, and/or one or more sagittal cross-section(s) 806 of the virtual model 800. Of course, other views are possible and can be included on the virtual model display 850. In some embodiments, the virtual model 800 may be interactive such that a user can manipulate the orientation or view of the virtual model 800 (e.g., rotate), change the depth of the displayed cross-sections, select and isolate specific bony structures, or the like.

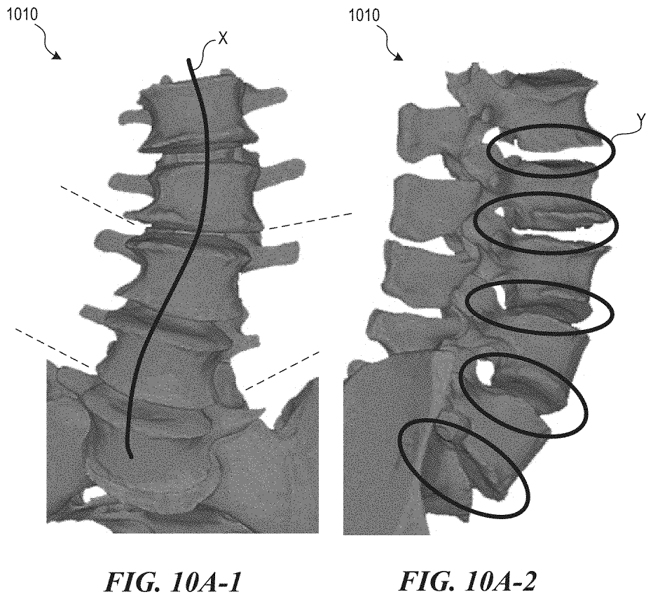

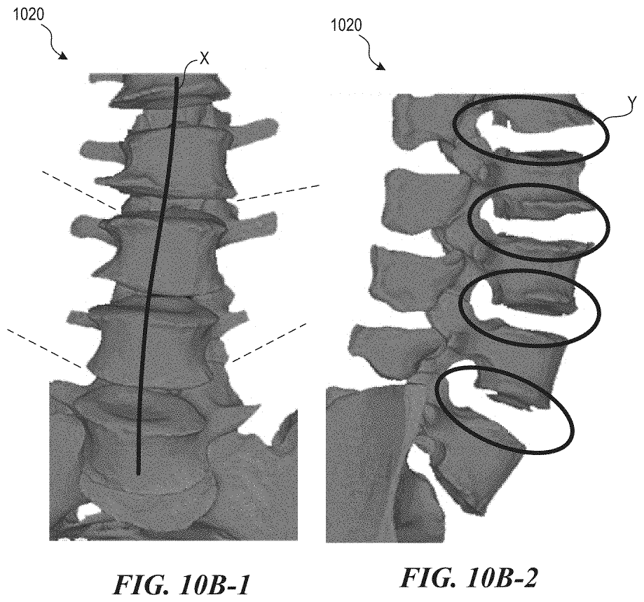

FIGS. 10A-1 and 10B-2 demonstrate an example of a virtual model of a patient's native anatomical configuration (e.g., as created in step 703 of the method 700) and a virtual model of the patient's corrected anatomical configuration (e.g., as created in step 704 of the method 700). In particular, FIGS. 10A-1 and 10A-2 are anterior and lateral views, respectively, of a virtual model 1010 showing a native anatomical configuration of a patient, and FIGS. 10B-1 and 10B-2 are anterior and lateral views, respectively, of a virtual model 1020 showing the corrected anatomical configuration for the same patient. Referring first to FIG. 10A-1, the anterior view of the virtual model 1010 illustrates the patient has abnormal curvature (e.g., scoliosis) of their spinal column. This is marked by line X, which follows a rostral-caudal axis of the spinal column. Referring next to FIG. 10A-1, the lateral view of the virtual model 1010 illustrates the patient has collapsed discs or decreased spacing between adjacent vertebral endplates, marked by ovals Y. FIGS. 10B-1 and 10B-2 illustrate the corrected virtual model 1020 accounting for the abnormal anatomical configurations shown in FIGS. 10A-1 and 10A-2. For example, FIG. 10B-1, which is an anterior view of the virtual model 1020, illustrates the patient's spinal column having corrected alignment (e.g., the abnormal curvature has been reduced). This correction is shown by line X, which also follows a rostral-caudal axis of the spinal column. FIG. 10B-2, which is a lateral view of the virtual model 1020, illustrates the patient's spinal column having restored disc height (e.g., increased spacing between adjacent vertebral endplates), also marked by ovals Y. The lines X and the ovals Y are provided in FIGS. 10A-1-10B-2 to more clearly demonstrate the correction between the virtual models 1010 and 1020, and are not necessarily included on the virtual models generated in accordance with the present technology. FIGS. 10A-1 and 10B-1 also show access paths/instrument positions (illustrated in dashed lines) for bilateral decompression procedures. The physician can view the proposed access paths/instrument positions to determine whether to perform decompression steps before, during, and/or after spinal alignment.

FIG. 11 illustrates an example of a surgical plan 1000 (e.g., as generated in step 706 of the method 700). The surgical plan 1000 can include pre-operative patient metrics 1002, predicted post-operative patient metrics 1004, one or more patient images (e.g., the patient images 703 received as part of the patient data set), the virtual model 910 (which can be the model itself or one or more images derived from the model) of the patient's native anatomical configuration (e.g., pre-operative patient anatomy), and/or the virtual model 920 (which can be the model itself or one or more images derived from the model) of the patient's corrected anatomical configuration (e.g., predicted post-operative patient anatomy). The instruments and items disclosed herein can be designed based on the virtual model 910, intra-operative models, and/or the post-operative virtual model 920. The virtual model 920 of the predicted post-operative patient anatomy can optionally include one or more implants 1012 shown as implanted in the patient's spinal cord region to demonstrate how patient anatomy will look following the surgery, including decompression steps, site preparation, implantation, etc. Although four implants 1012 are shown in the virtual model 920, the surgical plan 1000 may include more or fewer implants 1012, including one, two, three, five, six, seven, eight, or more implants 1012.

The surgical plan 1000 can include additional information beyond what is illustrated in FIG. 11. For example, the surgical plan 1000 may include pre-operative instructions, operative instructions, and/or post-operative instructions. Operative instructions can include one or more specific procedures to be performed (e.g., PLIF, ALIF, TLIF, LLIF, DLIF, XLIF, etc.) and/or one or more specific targets of the operation (e.g., fusion of vertebral levels L1-L4, anchoring screw to be inserted in lateral surface of L4, etc.). Although the surgical plan 1000 is demonstrated in FIG. 11 as a visual report, the surgical plan 1000 can also be encoded in computer-executable instructions that, when executed by a processor connected to a computing device, cause the surgical plan 1000 to be displayed by the computing device. In some embodiments, the surgical plan 1000 may also include machine-readable operative instructions for carrying out the surgical plan. For example, the surgical plan can include operative instructions for a robotic surgical platform to carry out one or more steps of the surgical plan 1000.

FIG. 12 provides a series of images illustrating an example of a patient surgical plan report 1100 that includes the surgical plan 1000 and that may be transmitted to a surgeon for review and approval (e.g., as transmitted in step 708 of the method 700). The surgical plan report 1100 can include a multi-page report detailing aspects of the surgical plan 1000. For example, the multi-page report may include a first page 1101 demonstrating an overview of the surgical plan 1000 (e.g., as shown in FIG. 11), a second page 1102 illustrating patient images (e.g., such as the patient images 703 received in step 702 and shown in FIG. 9D), a third page 1103 illustrating an enlarged view of the virtual model of the corrected anatomical configuration (e.g., the virtual model 920 shown in FIG. 11), and a fourth page 1104 prompting the surgeon to either approve or reject the surgical plan 1000. Of course, additional information about the surgical plan can be presented with the report 1100 in the same or different formats. In some embodiments, if the surgeon rejects the surgical plan 1000, the surgeon can be prompted to provide feedback regarding the aspects of the surgical plan 1000 the surgeon would like adjusted.

The patient surgical plan report 1100 can be presented to the surgeon on a digital display of a computing device (e.g., the client computing device 302 shown in FIG. 3 or the computing device 602 shown in FIG. 6). In some embodiments, the report 1100 is interactive and the surgeon can manipulate various aspects of the report 1100 (e.g., adjust views of the virtual model, zoom-in, zoom-out, annotate, etc.). However, even if the report 1100 is interactive, the surgeon generally cannot directly change the surgical plan 1000. Rather, the surgeon may provide feedback and suggested changes to the surgical plan 1000, which can be sent back to the computing system that generated the surgical plan 1000 for analysis and refinement.

FIG. 13A illustrates an example of a patient-specific implant 1200 (e.g., as designed in step 716 and manufactured in step 718 of the method 700), and FIG. 13B illustrates the implant 1200 implanted in the patient. The implant 1200 can be any orthopedic or other implant specifically designed to induce the patient's body to conform to the previously identified corrected anatomical configuration. In the illustrated embodiment, the implant 1200 is an vertebral interbody device having a first (e.g., upper) surface 1202 configured to engage an inferior endplate surface of a superior vertebral body and a second (e.g., lower) surface 1204 configured to engage a superior endplate surface of an inferior vertebral body. The first surface 1202 can have a patient-specific topography designed to match (e.g., mate with) the topography of the inferior endplate surface of the superior vertebral body to form a generally gapless interface therebetween. Likewise, the second surface 1204 can have a patient-specific topography designed to match or mate with the topography of the superior endplate surface of the inferior vertebral body to form a generally gapless interface therebetween. The implant 1200 may also include a recess 1206 or other feature configured to promote bony ingrowth. Because the implant 1200 is patient-specific and designed to induce a geometric change in the patient, the implant 1200 is not necessarily symmetric, and is often asymmetric. For example, in the illustrated embodiment, the implant 1200 has a non-uniform thickness such that a plane defined by the first surface 1202 is not parallel to a central longitudinal axis A of the implant 1200. Of course, because the implants described herein, including the implant 1200, are patient-specific, the present technology is not limited to any particular implant design or characteristic. Additional features of patient-specific implants that can be designed and manufactured in accordance with the present technology are described in U.S. patent application Ser. Nos. 16/987,113 and 17/100,396, the disclosures of which are incorporated by reference herein in their entireties.

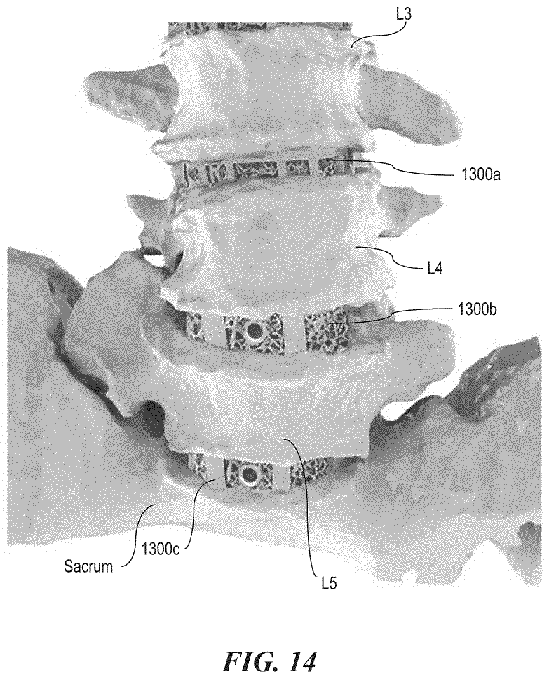

The patient-specific medical procedures described herein can involve implanting more than one patient-specific implant into the patient to achieve the corrected anatomical configuration (e.g., a multi-site procedure). FIG. 14, for example, illustrates a lower spinal cord region having three patient-specific implants 1300a-1300c implanted at different vertebral levels. More specifically, a first implant 1300a is implanted between the L3 and L4 vertebral bodies, a second implant 1300b is implanted between the L4 and L5 vertebral bodies, and a third implant 1300c is implanted between the L5 vertebral body and the sacrum. Together, the implants 1300a-c can cause the patient's spinal cord region to assume the previously identified corrected anatomical configuration (e.g., transforming the patient's anatomy from its pre-operative diseased configuration to the post-operative optimized configuration). In some embodiments, more or fewer implants are used to achieve the corrected anatomical configuration. For example, in some embodiments one, two, four, five, six, seven, eight, or more implants are used to achieve the corrected anatomical configuration. In embodiments involving more than one implant, the implants do not necessarily have the same shape, size, or function. In fact, the multiple implants will often have different geometries and topographies to correspond to the target vertebral level at which they will be implanted. As also shown in FIG. 14, the patient-specific medical procedures described herein can involve treating the patient at multiple target regions (e.g., multiple vertebral levels).

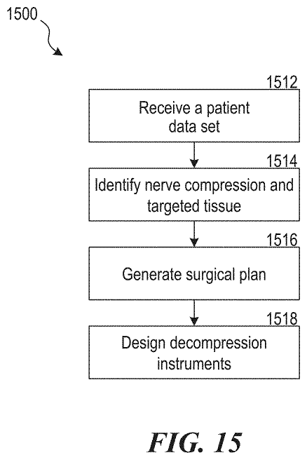

FIG. 15 is a flow diagram illustrating a method 1500 for providing patient-specific decompression procedures, according to an embodiment. The method 1500 can include steps discussed in connection with other methods disclosed herein, such as method 500 discussed in connection with FIG. 5 and method 700 discussed in connection with FIG. 7. In general, the method 1500 can be used to identify tissue compression (e.g., nerve compression), or other nerve compression information that is used to develop a surgical plan for providing nerve decompression with or without anatomical corrections.

At block 1512, the system can receive patient data including one or more images of the patient's spinal region showing the patient's anatomy. The system can also include pain information, disability information, questionnaire information, or other information associated with nerve compression. The system can provide diagnostic recommendations to the physician to perform additional tests to identify the location of nerve compression. In some procedures, the system can analyze images of the patient's anatomy and identify candidate levels of nerve compression. The system can provide diagnostic protocols to the physician to perform patient examinations to identify candidate levels likely to cause nerve compression. Additional imaging can be performed to obtain images showing, for example, stenosis, bulging discs, or other information.

Block 1512 can also include comparisons of the patient data to reference patient data sets in order to identify similar patients in the reference patient data sets. The comparisons can be based on, for example, pain scores, anatomical similarities, bone quality, foramen dimensions, location and position of nerve tissue, or the like. In some embodiments, the system can correlate pain data with one or more candidate nerve compression sites to identify candidate nerve compression sites with a high confidence score of nerve compression. Images can be segmented to identify bone, soft tissue (e.g., cartilage, nerve tissue, or the like) and can analyze historical data to identify candidate nerve compression sites. In some embodiments, pain reduction score(s) can be predicted for each surgical step or group of surgical steps of a surgical plan. For example, a first pain reduction score can be predicted for removing tissue associated with nerve compression. A second pain reduction score for implantation of at least one implant can be predicted. The surgical plan is generated to keep pain reduction scores at or above threshold levels (e.g., 50% reduction, 60% reduction, 70% reduction, 80% reduction, 90% reduction, 95% reduction, or 100% reduction). The pain reduction can be based on patient questionnaires, historical data, etc.

At block 1514, nerve compression sites and targeted tissue can be identified. The nerve compression site can be identified by segmenting patient images to identify anatomical features, tissue types, and other information indicative of nerve compression. In some procedures, one or more candidate nerve compression sites are identified using segmented patient images, pain scores, disability scores, questionnaires (e.g., questionnaires indicating locations of pain, severity of pain, location of disability, severity of disability, etc.), and other information. The system can correlate pain and disability data with one or more candidate nerve compression sites to identify candidate nerve compression sites as sites of nerve compression. In some embodiments, the nerve compression and target at least partially contributing to the nerve compression are identified. The nerve compression can be identified based on abnormal anatomical features adjacent nerve tissue, bulging discs, impinging tissue, patient feedback (e.g., patient identified areas of pain), or the like. In some embodiment, the patient data set can be compared to prior patient data sets to identify similarities based on anatomy, pain, symptoms, etc. Matching sets of data can then be identified correlating the pain or disability to the anatomical features along the spine. The identified nerve compression can be based, at least in part, on one or more surgical corrections to the anatomy. This allows the system to predict whether nerve compression will be altered due to the corrected anatomical configuration discussed above.

At block 1516, the system can generate a surgical plan that includes one or more decompression steps, implantation steps, or combinations thereof. For virtual simulations, the system can generate a virtual model of a spinal region to illustrate compression along the spine. The system can generate a decompressed virtual model of the spine in which one or more regions of nerve compression have been reduced or eliminated. The system can simulate spinal motion related to spinal nerve compression based on different loading conditions.

The virtual model can be three-dimensional model bones, soft tissue, connective tissue, implants, instruments, and other features of interest. Is some embodiments, the virtual model can be a computer aided (CAD) design model generated based on the patient images. Material properties can be assigned to different types of tissue, such as Bony tissue, cartilage, nerve tissue, etc.

A physician can modify the motion based on the activities performed by the user, user's age, or the like. The system can generate a surgical decompression plan that includes images of the decompressed virtual model of the spinal region in which nerve tissue is decompressed, predicted pain reduction scores, identification of target levels for decompression, and/or other information associated with decompression. A physician can analyze the images of the decompressed virtual model to determine whether anatomical corrections associated with the decompression should be performed. The pain reduction scores can be based on reference pain reduction scores from prior patients. The physician can modify the amount of targeted tissue to be removed to adjust the score. A physician can balance the structural integrity of vertebral bodies with bone removed from vertebral bodies for decompression.

The system can generate a surgical decompression plan that includes one or more images of the decompressed virtual model of the spinal region in which nerve tissue is decompressed, one or more predicted metrics (e.g., pain reduction scores, mobility increase, etc.) for the surgical decompression plan, and/or identification of target sites and/or levels for decompression. The physician can modify and approve the surgical decompression plan. The system can then design one or more instruments based on the surgical decompression plan.

The surgical plan can indicate safe working zones in which the instruments can be positioned to perform a decompression step. With reference to FIG. 2A, a safe working zone 72 is illustrated as a triangle shaped zone illustrating to the physician the desired position of the distal end of the instrument. The safe working zone is spaced apart from the spinal cord 56 by the ligamentum flavum 76 to ensure that the spinal cord is protected. The system can illustrate the enlargement of the working zone 72, as shown in FIGS. 2B and 2C, for different decompression steps to allow the physician to plan for the surgical procedure and manipulation of the surgical instruments. In some embodiments, any of the operations described with respect to block 1516 can be incorporated into step 706 of the method 700, e.g., to generate surgical plans that address multiple pathological issues in a patient and/or that recommend multiple complementary procedures (e.g., decompression and fusion, etc.).

At block 1518 of FIG. 15, the system can design decompression instruments for performing the decompression plan. As discussed above with respect to FIGS. 2A-2C, the distal heads can have atraumatic features or regions configured to face nerve tissue when the distal head alters tissue at the site. The instrument can be used to perform tasks while limiting or preventing trauma to nerve tissue. In some embodiments, the instruments have elongated bodies designed to extend along access paths to enable planned surgical acts to be performed. For example, the instrument 50a of FIG. 2A has an elongate body that extends along a long axis 51 that can lie parallel to or along an access path 54. In some embodiments, any of the operations described with respect to block 1518 can be incorporated into step 716 of the method 700, e.g., such that the method 700 can design decompression instruments in addition to other patient-specific components for complementary procedures.

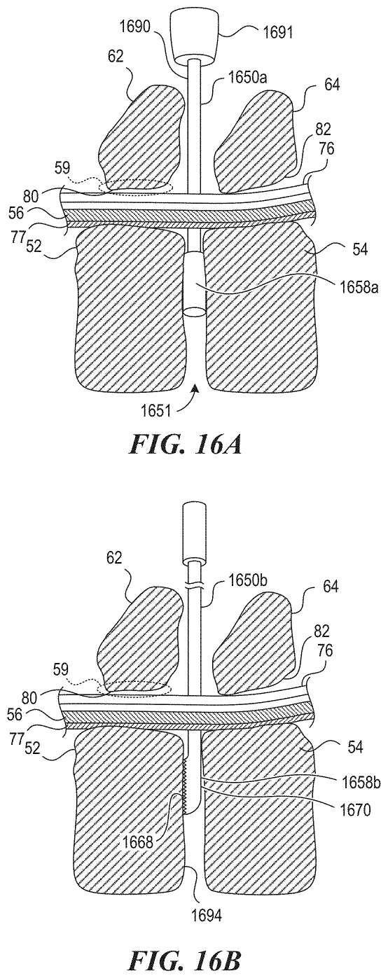

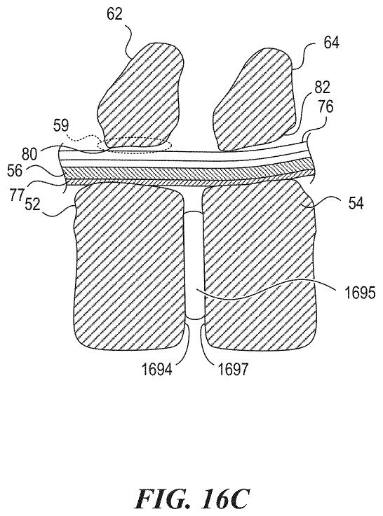

FIGS. 16A-16C are detailed side views of distal ends of instruments positioned along a spine prior to preforming the steps discussed in FIGS. 2A-2B, according to an embodiment. FIG. 16A shows a patient-specific instrument 1650a that can include a reamer head 1658a. The instrument 1650a can include a shaft 1690 extending between a handle 1691 and the reamer head 1658a. The configuration of the shaft 1690 can be selected based on the anatomical features of the patient. The description of instrument 50c of FIG. 2C applies equally to the instrument 1650a unless indicated otherwise.

The instrument 1650a can be used to remove tissue at least partially contributing to nerve compression and can be used to repair the intervertebral disc space for receiving an implant. FIG. 16A shows the reamer head 1658a abrading the exposed vertebral endplates to, for example, roughen the endplate surfaces, flatten or shape bony surfaces, or the like. The reamer head 1658a can be dimensioned to fit in the disc space 1651 and have, for example, a cylindrical shape, a wavy shape, a conical shape, a spheroid shaped, or other suitable shape for being placed within the disc space 1651.

FIG. 16B shows an instrument 1650b including a distal portion 1658b having a roughened or textured surface 1668 and an atraumatic surface 1670. The textured surface 1668 can be used to roughen one vertebral endplate while the opposing atraumatic surface 1670 faces the opposing endplate. The textured surface 1668 can be configured to shape the vertebral surface 1694 of the vertebra 52 already modified by the instrument 1650a of FIG. 16A. This allows each instrument to be designed based on the prior steps.

FIG. 16C shows an implant 1695 contacting the modified endplate surfaces 1694, 1697. The implant 1695 can be designed based on the modified vertebral endplates rather than the pre-operative endplates for flexibility when designing the surgical plan. Additionally, one or more additional surgical steps can be performed during and/or after implantation of the implant 1695. In some embodiments, the implant 1695 is a fixed height cage. After implantation, a decompression procedure can be performed to remove target tissue 59 to alleviate predicted post-operative nerve compression. The instruments can include one or more radiopaque markers to facilitate visualization under, for example, fluoroscopy, or other visualization techniques. In some procedures, the implant 1695 is expandable. During or after expansion, one or more decompression steps can be performed based on real-time visualization of the patient's spine. Accordingly, decompression steps can be performed before, during, and/or after placement of the implant 1695.

Virtual models can be used to simulate one or more surgical steps to, for example, design surgical kits, design instruments, design implants, generate surgical plans, develop training protocols, etc. For example, virtual models of the anatomy of FIGS. 16A-16C can be used to design the tools 1650a, 1650b and implant 1695, as well as other items disclosed herein.

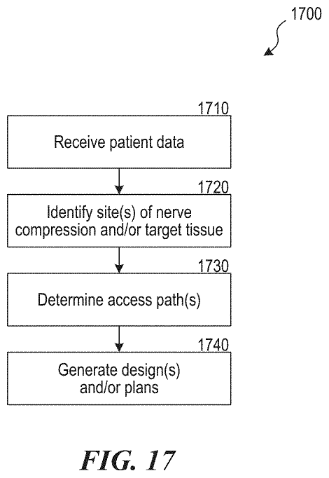

FIG. 17 shows a method 1700 for designing instruments. At block 1710, patient data is received. The patient data can include one or more images of the patient's showing the patient's anatomy, pain data, etc. At block 1720, at least one site of nerve compression and/or target tissued contributing to the nerve compression can be identified based on the received patient data. At block 1730, one or more access paths for surgically accessing the targeted tissue can be determined. At block 1740, a patient-specific instrument configured to access the targeted tissue via the access path and to perform a decompression step on the targeted tissue can be designed. In some embodiments, a personalized surgical plan for implanting one or more patient-specific interbody devices for achieving a target corrected spinal configuration for a segment of patient's spine can be generated. Additionally, a decompression plan for utilizing the at least one patient-specific instruments in conjunction with implantation of the one or more patient-specific interbody devices can be generated.

The computer-implemented methods 500 (FIG. 5), 700 (FIG. 7), 1500 (FIG. 15), 1700 (FIG. 17) can be used to generate a surgical plan with one or more spinal corrections for the patient's pre-operative spine for achieving a target corrected spinal configuration based on the patient data. The system can predict and compensate for post-operative nerve compression associated with the spinal corrections. The predicting and compensating can include comparing a pre-operative configuration of vertebrae of the patent to a post-operative target configuration of vertebrae of the patent, identifying post-operative nerve compression that exceeds a threshold level based on the comparison and that are caused by the correction, and generating a decompression plan to compensate for the identified post-operative nerve compression. The method 500 (FIG. 5), 700 (FIG. 7), 1500 (FIG. 15), 1700 (FIG. 17) can identify nerve compression and target tissued at least partially contributing to the nerve compression based on the received patient data and design at least one patient-specific instrument (e.g., powered instruments, manual instruments, etc.) configured to perform one or more decompression steps based on the one or more spinal corrections.

The method 1700 can generate a personalized surgical plan for implanting one or more patient-specific interbody devices for achieving a target corrected spinal configuration for a spinal segment. The method 1700 can predict and compensate for the post-operative nerve compression by, for example, simulating the one or more spinal corrections by moving features of a three-dimensional virtual model of the patient's anatomy and identifying compression of nerve tissue caused by the movement of features of the three-dimensional virtual model and modifying the surgical plan to reduce the identified compression of nerve tissue. In some embodiments, predicting and compensating for the post-operative nerve compression include, for example, virtually simulating nerve compression caused by one or more spinal corrections and generating a decompression plan based on the virtual simulation of nerve tissue.

The method 1700 of FIG. 17 can include designing a set of patient-specific decompression instruments based on the decompression plan and predicting pre-operative, intra-operative and/or post-operative nerve compression associated with the surgical plan and determine, based on the predicted intra-operative and/or post-operative nerve compression, whether to modify the surgical plan, recommend a decompression procedure, or both. In response to receiving user approval of the decompression procedure, the method 1700 can include designing one or more patient-specific decompression instruments. One or more additional post-operative adverse effects can be predicted and/or compensated for by at least one of modifying the surgical plan or generating additional surgical steps. Pre-operative nerve compression causing pain and/or discomfort can be identified based on pain data of the patient. The nerve compression can remain post-operatively when the one or more spinal corrections do not cause significant reduction of the nerve compression. A decompression procedure can be performed reduce or eliminate the post-operative spinal nerve compression. Post-operative pain data for training a machine-learning model can be collected and then used to predict the post-operative nerve compression. One or more patient-specific instruments can be designed based on a pre-operative spinal configuration, intra-operative spinal configuration, and/or the corrected spinal configuration. A level-by-level nerve compression score for the surgical plan can be displayed to a user.

In some design protocols, the decompression instrument can be designed to fit through the cannula or other delivery instrument. The distal portion of the instrument can be designed to move throughout a working space identified at the treatment site. The display can also be designed to limit or prevent traumatizing or damaging adjacent nontargeted tissue. In some embodiments, the distal portion can be designed to slide between tissue, pierce tissue, or otherwise be moved at a surgical site.

In addition to designing patient-specific medical care based off reference patient data sets, the systems and methods of the present technology may also design patient-specific medical care based off disease progression for a particular patient (e.g., a human adult, a human child, etc.). In some embodiments, the present technology therefore includes software modules (e.g., machine learning models or other algorithms) that can be used to analyze, predict, and/or model disease progression for a particular patient. The machine learning models can be trained based off a plurality of reference patient data sets that includes, in addition to the patient data described with respect to FIG. 3, disease progression metrics for each of the reference patients. The progression metrics can include measurements for disease metrics over a period of time. Suitable metrics may include spinopelvic parameters (e.g., lumbar lordosis, pelvic tilt, sagittal vertical axis (SVA), Cobb angle, coronal offset, etc.), disability scores, functional ability scores, flexibility scores, VAS pain scores, or the like. The progression of the metrics for each reference patient can be correlated to other patient information for the specific reference patient (e.g., age, sex, height, weight, activity level, diet, etc.).

In some embodiments, the present technology includes a disease progression module that includes an algorithm, machine learning model, or other software analytical tool for predicting disease progression in a particular patient. The disease progression module can be trained based on reference patient data sets that includes patient information (e.g., age, sex, height, weight, activity level, diet) and disease metrics (e.g., diagnosis, spinopelvic parameters such as lumbar lordosis, pelvic tilt, sagittal vertical axis, Cobb angle, coronal offset, etc., disability scores, functional ability scores, flexibility scores, VAS pain scores, etc.). The disease metrics can include values over a period of time. For example, the reference patient data may include values of disease metrics on a daily, weekly, monthly, bi-monthly, yearly, or other basis. By measuring the metrics over a period of time, changes in the values of the metrics can be tracked as an estimate of disease progression and correlated to other patient data.

In some embodiments, the disease progression module can therefore estimate the rate of disease progression for a particular patient. The progression may be estimated by providing estimated changes in one or more disease metrics over a period of time (e.g., X % increase in a disease metric per year). The rate can be constant (e.g., 5% increase in pelvic tilt per year) or variable (e.g., 5% increase in pelvic tilt for a first year, 10% increase in pelvic tilt for a second year, etc.). In some embodiments, the estimated rate of progression can be transmitted to a surgeon or other healthcare provider, who can review and update the estimate, if necessary.

As a non-limiting example, a particular patient who is a fifty-five-year-old male may have a SVA value of 6 mm. The disease progression module can analyze patient reference data sets to identify disease progression for individual reference patients have one or more similarities with the particular patient (e.g., individual patients of the reference patients who have an SVA value of about 6 mm and are approximately the same age, weight, height, and/or sex of the patient). Based on this analysis, the disease progression module can predict the rate of disease progression if no surgical intervention occurs (e.g., the patient's VAS pain scores may increase 5%, 10%, or 15% annually if no surgical intervention occurs, the SVA value may continue to increase by 5% annually if no surgical intervention occurs, etc.).

The systems and methods described herein can also generate models/simulations based on the estimated rates of disease progression, thereby modeling different outcomes over a desired period of times. Additionally, the models/simulations can account for any number of additional diseases or condition to predict the patient's overall health, mobility, or the like. These additional diseases or conditions can, in combination with other patient health factors (e.g., height, weight, age, activity level, etc.) be used to generate a patient health score reflecting the overall health of the patient. The patient health score can be displayed for surgeon review and/or incorporated into the estimation of disease progression. Accordingly, the present technology can generate one or more virtual simulations of the predicted disease progression to demonstrate how the patient's anatomy is predicted to change over time. Physician input can be used to generate or modify the virtual simulation(s). The present technology can generate one or more post-treatment virtual simulations based on the received physician input for review by the healthcare provider, patient, etc.

In some embodiments, the present technology can also predict, model, and/or simulate disease progression based on one or more potential surgical interventions. For example, the disease progression module may simulate what a patient's anatomy may look like 1, 2, 5, or 10 years post-surgery for several surgical intervention options. The simulations may also incorporate non-surgical factors, such as patient age, height, weight, sex, activity level, other health conditions, or the like, as previously described. Based on these simulations, the system and/or a surgeon can select which surgical intervention is best suited for long-term efficacy. These simulations can also be used to determine patient-specific corrections that compensate for the projected diseases progression.

Accordingly, in some embodiments, multiple disease progression models (e.g., two, three, four, five, six, or more) are simulated to provide disease progression data for several different surgical intervention options or other scenarios. For example, the disease progression module can generate models that predict post-surgical disease progression for each of three different surgical interventions. A surgeon or other healthcare provider can review the disease progression models and, based on the review, select which of the three surgical intervention options is likely to provide the patient with the best long-term outcome. Of course, selecting the optimal intervention can also be fully or semi-automated, as described herein.

Based off of the modeled disease progression, the systems and methods described herein can also (i) identify the optimal time for surgical intervention, and/or (ii) identify the optimal type of surgical procedure for the patient. In some embodiments, the present technology therefore includes an intervention timing module that includes an algorithm, machine learning model, or other software analytical tool for determining the optimal time for surgical intervention in a particular patient. This can be done, for example, by analyzing patient reference data that includes (i) pre-operative disease progression metrics for individual reference patients, (ii) disease metrics at the time of surgical intervention for individual reference patients, (iii) post-operative disease progression metrics for individual reference patients, and/or (iv) scored surgical outcomes for individual reference patients. The intervention timing module can compare the disease metrics for a particular patient to the reference patient data sets to determine, for similar patients, the point of disease progression at which surgical intervention produced the most favorable outcomes.

As a non-limiting example, the reference patient data sets may include data associated with reference patients' sagittal vertical axis. The data can include (i) sagittal vertical axis values for individual patients over a period of time before surgical intervention (e.g., how fast and to what degree the sagittal vertical axis value changed), (ii) sagittal vertical axis of the individual patients at the time of surgical intervention, (iii) the change in sagittal vertical axis after surgical intervention, and (iv) the degree to which the surgical intervention was successful (e.g., based on pain, quality of life, or other factors). Based on the foregoing data, the intervention timing module can, based on a particular patient's sagittal vertical axis value, identify at which point surgical intervention will have the highest likelihood of producing the most favorable outcome. Of course, the foregoing metric is provided by way of example only, and the intervention timing module can incorporate other metrics (e.g., lumbar lordosis, pelvic tilt, sagittal vertical axis, Cobb angle, coronal offset, disability scores, functional ability scores, flexibility scores, VAS pain scores) instead of or in combination with sagittal vertical axis to predict the time at which surgical intervention has the highest probability of providing a favorable outcome for the particular patient.

The intervention timing module may also incorporate one or more mathematical rules based on value thresholds for various disease metrics. For example, the intervention timing module may indicate surgical intervention is necessary if one or more disease metrics exceed a predetermined threshold or meet some other criteria. Representative thresholds that indicate surgical intervention may be necessary include SVA values greater than 7 mm, a mismatch between lumbar lordosis and pelvic incidence greater than 10 degrees, a Cobb angle of greater than 10 degrees, and/or a combination of Cobb angle and LL/PI mismatch greater than 20 degrees. Of course, other threshold values and metrics can be used; the foregoing are provided as examples only and in no way limit the present disclosure. In some embodiments, the foregoing rules can be tailored to specific patient populations (e.g., for males over 50 years of age, an SVA value greater than 7 mm indicates the need for surgical intervention). If a particular patient does not exceed the thresholds indicating surgical intervention is recommended, the intervention timing module may provide an estimate for when the patient's metrics will exceed one or more thresholds, thereby providing the patient with an estimate of when surgical intervention may become recommended. In some embodiments, a sequence for using instruments or performing surgical steps can be based on at least one procedure threshold. The procedure threshold can include a safety threshold, a time threshold, and/or a predicted outcome threshold. The procedure threshold can be, for example, a likelihood of an adverse event, a predicted time to complete surgical step(s), a predicted outcome, combination thereof (e.g., weighted or unweighted), etc. Different sequences of surgical steps can be simulated to identify a sequence of surgical steps that meet or exceed procedure thresholds. Any number of procedure thresholds can be used to develop a surgical plan.

The present technology may also include a treatment planning module that can identify the optimal type of surgical procedure for the patient based on the disease progression of the patient. The treatment planning module can be an algorithm, machine learning model, or other software analytical tool trained or otherwise based on a plurality of reference patient data sets, as previously described. The treatment planning module may also incorporate one or more mathematical rules for identifying surgical procedures. As a non-limiting example, if a LL/PI mismatch is between 10 and 20 degrees, the treatment planning module may recommend an anterior fusion surgery, but if the LL/PI mismatch is greater than 20 degrees, the treatment planning module may recommend both anterior and posterior fusion surgery. As another non-limiting example, if a SVA value is between 7 mm and 15 mm, the treatment planning module may recommend posterior fusion surgery, but if the SVA is above 15 mm, the treatment planning module may recommend both posterior fusion surgery and anterior fusion surgery. Of course, other rules can be used; the foregoing are provided as examples only and in no way limit the present disclosure.

Without being bound by theory, incorporating disease progression modeling into the patient-specific medical procedures described herein may even further increase the effectiveness of the procedures. For example, in many cases it may be disadvantageous operate after a patient's disease progresses to an irreversible or unstable state. However, it may also be disadvantageous to operate too early, before the patient's disease is causing symptoms and/or if the patient's disease may not progress further. The disease progression module and/or the intervention timing module can therefore help identify the window of time during which surgical intervention in a particular patient has the highest probability of providing a favorable outcome for the patient.

### EXAMPLES

1. A computer-implemented method comprising:
receiving patient data for a patient, the patient data including one or more images of the patient's spinal region showing the patient's anatomy;
identifying at least one site of nerve compression and target tissue at least partially contributing to the nerve compression based on the received patient data; and
designing at least one patient-specific instrument configured to access the target tissue and perform a decompression step on the target tissue.

2. The computer-implemented method of example 1, further comprising:
determining an access path for surgically accessing the target tissue, wherein the at least one patient-specific instrument is designed to access the target tissue using the access path;
generating a personalized surgical plan for implanting one or more patient-specific interbody devices for achieving a target corrected spinal configuration for a segment of the patient's spine; and
generating a decompression plan for utilizing the at least one patient-specific instrument in conjunction with implantation of the one or more patient-specific interbody devices.

3. The computer-implemented method of any one or a combination of examples 1 or 2, further comprising:
generating a three-dimensional virtual model representing the patient's spinal region; and
designing, using the three-dimensional virtual model, at least one surgical site preparation instrument configured to prepare an implantation site for an implant.

4. The computer-implemented method of any one or a combination of examples 3, further comprising designing the at least one patient-specific instrument and the at least one surgical site preparation instrument to be passed through a cannula positioned in the patient or an incision in the patient.

5. The computer-implemented method of any one or a combination of examples 1-3, further comprising:
designing the at least one patient-specific instrument to be delivered through a first cannula positioned in the patient; and
designing the at least one surgical site preparation instrument to be delivered through a second cannula positioned in the patient and spaced apart from the first cannula.

6. The computer-implemented method of any one or a combination of examples 1-5, wherein designing the at least one patient-specific instrument includes:
determining an access path to the target tissue;
designing a distal head with an atraumatic region configured to face nerve tissue when the distal head removes the target tissue; and
designing an elongated body connected to the distal head and configured to extend along the access path to position the distal head at the at least one site and enable removal of the target tissue.

7. The computer-implemented method of example 6, wherein the distal head is a debulking head, a rongeur head, a cutting head, or a reamer.

8. The computer-implemented method of any one or a combination of examples 1-7, further comprising determining a safe working zone for performing the decompression step,
wherein designing the at least one patient-specific instrument includes configuring a distal end of the at least one patient-specific instrument to remain in the safe working zone when performing the decompression step.

9. The computer-implemented method of any one or a combination of examples 1-9, wherein the patient data further includes pain data, the method comprising:
identifying one or more candidate nerve compression sites, and
correlating the pain data with the one or more candidate nerve compression sites to identify at least one of the candidate nerve compression sites as the at least one site of nerve compression.

10. The computer-implemented method of any one or a combination of examples 1-9, wherein designing the at least one patient-specific instrument includes:
determining a sequence of steps to remove the target tissue; and
designing a plurality of patient-specific instruments each configured to perform at least one of the steps in the sequence.

11. The computer-implemented method of any one or a combination of examples 1-10, further comprising:
generating a virtual model of the patient's spinal region;
simulating, using the virtual model, spinal motion to determine motion related spinal nerve compression based on different loading conditions; and
generating a surgical decompression plan including
   one or more images of the virtual model of the spinal region in which nerve tissue is decompressed,
   one or more pain reduction scores for the surgical decompression plan, and/or
   identification of one or more target levels for decompression.

12. The computer-implemented method of example 11, wherein the surgical decompression plan identifies one or more safe working zones in which the at least one patient-specific instrument can be positioned to perform the decompression step.

13. The computer-implemented method of any one or a combination of examples 1-12, further comprising generating a surgical decompression plan that identifies tissues, tissue margins, and/or nerve compression sites.

14. The computer-implemented method of any one or a combination of examples 1-13, further comprising:
receiving user input for hand movement;
determining one or more manipulation parameters based on the user input; and
designing the at least one patient-specific instrument based on the determined one or more manipulation parameters.

15. The computer-implemented method of any one or a combination of examples 1-14, further comprising:
simulating a surgical procedure using hand input devices operated by the user to generate the user input, wherein the user input includes one or more of at least one range of motion of one or both hands of the user, the user's strength, and/or the user's manual instrument positioning accuracy.

16. The computer-implemented method of any one or a combination of examples 1-15, wherein the at least one patient-specific instrument includes a set of instruments each configured to remove tissue from the target site.

17. The computer-implemented method of any one or a combination of examples 1-16, further comprising:
determining a sequence for using the instruments in the set based on at least one procedure threshold.

18. The computer-implemented method of example 17, wherein the at least one procedure threshold includes a safety threshold, a time threshold, and/or predicted outcome threshold.

19. A system for a patient-specific spinal decompression procedure, the system comprising:
one or more processors; and
a memory storing instructions that, when executed by the one or more processors, cause the system to perform operations comprising:
   receiving patient data for a patient, the patient data including one or more images of the patient's anatomy;
   identifying at least one site of nerve compression and target tissued at least partially contributing to the nerve compression based on the received patient data;
   designing at least one patient-specific instrument configured to access the target tissue and perform a decompression step on the target tissue.

20. The system of example 19, wherein the operations further include
generating a personalized surgical plan for implanting one or more patient-specific interbody devices for achieving a target corrected spinal configuration for a segment of the patient's spine; and
generating a decompression plan for utilizing the at least one patient-specific instrument in conjunction with implantation of the one or more patient-specific interbody devices.

21. The system of any one or a combination of examples 10-20, wherein the operations further include designing the at least one patient-specific instrument to include:
designing a distal head with an atraumatic region configured to face nerve tissue when the distal head removes the target tissue from the at least one site; and
designing an elongated body connected to the distal head and configured to extend along an access path to position the distal head at the at least one site.

22. The system of example 21, wherein the distal head is a debulking head, a rongeur head, a cutting head, or a reamer.

23. The system of any one or a combination of examples 10-22, wherein the operations further include determining a safe working zone for performing the decompression step,
wherein designing the at least one patient-specific instrument includes configuring a distal end of the at least one patient-specific instrument to remain in the safe working zone when performing the decompression step.

24. The system of any one or a combination of examples 19-23, wherein the patient data further includes pain data, the system comprising:
identifying one or more candidate nerve compression sites, and
correlating the pain data with the one or more candidate nerve compression sites to identify at least one of the one or more candidate nerve compression sites as the at least one site of nerve compression.

25. The system of any one or a combination of examples 19-24, wherein designing the at least one patient-specific instrument includes:
determining a sequence of steps to remove the target tissue; and
designing a plurality of patient-specific instruments each designed based on the patient's anatomy.

26. The system of any one or a combination of examples 19-25, wherein the operations further include:
generating a virtual model of the spinal region to illustrate the spine;
simulating, via the virtual model, spinal motion to determine motion related spinal nerve compression based on different loading conditions; and
generating a surgical decompression plan including
   one or more images of the virtual model of the spinal region in which nerve tissue is decompressed,
   one or more predicted pain reduction scores for the surgical decompression plan, and/or
   identification of one or more target levels for decompression.

27. The system of any one or a combination of examples 19-26, wherein the operations further include:
receiving user input for hand movement;
determining one or more manipulation parameters based on the user input; and
designing the at least one patient-specific instrument based on the determined one or more manipulation parameters.

28. The system of any one or a combination of examples 19-27, wherein the operations further include:
simulating a surgical procedure using hand input devices operated by the user to
   generate the user input, wherein the user input includes one or more of
   at least one range of motion of one or both hands of the user,
   the user's strength, and/or
   the user's manual instrument positioning accuracy.

29. The system of any one or a combination of examples 19-28, wherein the at least one patient-specific instrument includes a set of instruments each configured to remove tissue from the target site.

30. The system of any one or a combination of examples 19-29, wherein the operations further include:
determining a sequence for using the instruments in the set based on at least one procedure threshold.

31. The system of any one or a combination of examples 19-30, wherein the at least one procedure threshold includes a safety threshold, a time threshold, and/or predicted outcome threshold.

32. A computer-readable storage medium storing instructions that, when executed by a computing system, cause the computing system to perform operations comprising:
receiving patient data for a patient, the patient data including one or more images of the patient's spinal region showing the patient's anatomy;
identifying at least one site of nerve compression and target tissued at least partially contributing to the nerve compression based on the received patient data;
determining an access path for surgically accessing the target tissue; and
designing at least one patient-specific instrument configured to access the target tissue via the access path and to perform a decompression step on the target tissue.

33. A computer-readable storage medium of example 32, wherein the operations further include:
generating a personalized surgical plan for implanting one or more patient-specific interbody devices for achieving a target corrected spinal configuration for a segment of patient's spine; and
generating a decompression plan for utilizing the at least one patient-specific instruments in conjunction with implantation of the one or more patient-specific interbody devices.

34. A computer-implemented method comprising:
receiving patient data for a patient, the patient data including one or more images of anatomy of the patient;
determining a surgical plan with one or more spinal corrections for the patient's pre-operative spine for achieving a target corrected spinal configuration based on the patient data; and
predicting and compensating for post-operative nerve compression associated with the one or more spinal corrections.

35. The computer-implemented method of example 34, wherein predicting and compensating for the post-operative nerve compression includes:
comparing a pre-operative configuration of the patient's vertebrae to a post-operative target configuration of the vertebrae;
predicting post-operative nerve compression that is caused by the one or more spinal corrections and exceeds a threshold level based on the comparison; and
generating a decompression plan to compensate for the predicted post-operative nerve compression.

36. The computer-implemented method of any one or a combination of examples 34-35, further comprising:
predicting nerve compression; and
designing at least one patient-specific instrument configured to perform one or more decompression steps based on the predicted nerve compression.

37. The computer-implemented method of any one or a combination of examples 34-36, further comprising:
generating a personalized surgical plan for implanting one or more patient-specific interbody devices for achieving the target corrected spinal configuration for a segment of patient's spine.

38. The computer-implemented method of any one or a combination of examples 34-37, wherein predicting and compensating for the post-operative nerve compression includes:
simulating the one or more spinal corrections by moving features of a three-dimensional virtual model of the patient's anatomy;
identifying compression of nerve tissue caused by the movement of the features of the three-dimensional virtual model; and
modifying the surgical plan to reduce the identified compression of nerve tissue.

39. The computer-implemented method of any one or a combination of examples 34-38, wherein predicting and compensating for the post-operative nerve compression includes:
virtually simulating nerve compression caused by the one or more spinal corrections; and
generating a decompression plan based on the virtual simulation of nerve tissue.

40. The computer-implemented method of any one or a combination of examples 34-39, further comprising designing a set of patient-specific decompression instruments based on the surgical plan.

41. The computer-implemented method of any one or a combination of examples 34-40, further comprising:
predicting intra-operative and/or post-operative nerve compression associated with the surgical plan; and
determining, based on the predicted intra-operative and/or the post-operative nerve compression, whether to modify the surgical plan, recommend a decompression procedure, or both.

42. The computer-implemented method of any one or a combination of examples 34-41, further comprising in response to receiving user approval of a decompression procedure of the surgical plan, designing one or more patient-specific decompression instruments.

43. The computer-implemented method of any one or a combination of examples 34-42, further comprising predicting and compensating for one or more additional post-operative adverse effects by at least one of modifying the surgical plan or generating additional surgical steps.

44. The computer-implemented method of any one or a combination of examples 34-43, further comprising:

identifying pre-operative nerve compression causing pain and/or discomfort based on pain data of the patient; and

predicting that the nerve compression will remain post-operatively when the one or more spinal corrections do not cause significant reduction of the nerve compression.

45. The computer-implemented method of any one or a combination of examples 34-44, further comprising collecting post-operative pain data for training a machine-learning model that was used to predict the post-operative nerve compression.

46. The computer-implemented method of any one or a combination of examples 34-45, further comprising designing one or more patient-specific instruments based on a pre-operative spinal configuration of the patient, a predicted intra-operative spinal configuration of the patient, and/or the target corrected spinal configuration of the patient.

47. The computer-implemented method of any one or a combination of examples 34-46, further comprising determining a level-by-level nerve compression score for the surgical plan for display to a user.

48. The computer-implemented method of any one or a combination of examples 34-47, further comprising generating a decompression plan for substantially eliminated targeted nerve compression.

49. The computer-implemented method of any one or a combination of examples 34-48, further comprising identifying targeted tissue that can be removed from the patient to achieve the elimination of the targeted nerve compression.

50. The computer-implemented method of any one or a combination of examples 34-48, further comprising predicting a pain reduction score for a decompression procedure.

51. The computer-implemented method of any one or a combination of examples 34-50, further comprising:
predicting a first pain reduction score for removing tissue associated with nerve compression according to the surgical plan, and
predicting a second pain reduction score for implantation of at least one implant according to the surgical plan.

52. The computer-implemented method of any one or a combination of examples 34-51, further comprising:
designing a surgical kit for performing the surgical plan, wherein the surgical kit includes one or more instruments and one or more implants.

53. The computer-implemented method of example 52, wherein the one or more instruments are designed based on the one or more implants; and the one or more implants are designed based on the one or more instruments.

54. The computer-implemented method of example 52, further comprising:
sending instructions to manufacture the kit; and
validating that the manufactured kit is complete.

The foregoing detailed description has set forth various embodiments of the devices and/or processes via the use of block diagrams, flowcharts, and/or examples. As one skilled in the art will appreciate, any of the software modules described previously may be combined into a single software module for performing the operations described herein. Likewise, the software modules can be distributed across any combination of the computing systems and devices described herein, and are not limited to the express arrangements described herein. Accordingly, any of the operations described herein can be performed by any of the computing devices or systems described herein, unless expressly noted otherwise.

Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In some embodiments, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a floppy disk, a hard disk drive, a CD, a DVD, a digital tape, a computer memory, etc.; and a transmission type medium such as a digital and/or an analog communication medium (e.g., a fiber optic cable, a waveguide, a wired communications link, a wireless communication link, etc.).

Those skilled in the art will recognize that it is common within the art to describe devices and/or processes in the fashion set forth herein, and thereafter use engineering practices to integrate such described devices and/or processes into data processing systems. That is, at least a portion of the devices and/or processes described herein can be integrated into a data processing system via a reasonable amount of experimentation. Those having skill in the art will recognize that a typical data processing system generally includes one or more of a system unit housing, a video display device, a memory such as volatile and non-volatile memory, processors such as microprocessors and digital signal processors, computational entities such as operating systems, drivers, graphical user interfaces, and applications programs, one or more interaction devices, such as a touch pad or screen, and/or control systems including feedback loops and control motors (e.g., feedback for sensing position and/or velocity; control motors for moving and/or adjusting components and/or quantities). A typical data processing system may be implemented utilizing any suitable commercially available components, such as those typically found in data computing/communication and/or network computing/communication systems.

The herein described subject matter sometimes illustrates different components contained within, or connected with, different other components. It is to be understood that such depicted architectures are merely examples, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermediate components. Likewise, any two components so associated can also be viewed as being “operably connected,” or “operably coupled,” to each other to achieve the desired functionality, and any two components capable of being so associated can also be viewed as being “operably couplable” to each other to achieve the desired functionality. Specific examples of operably couplable include but are not limited to physically mateable and/or physically interacting components and/or wirelessly interactable and/or wirelessly interacting components and/or logically interacting and/or logically interactable components.

The embodiments, features, systems, devices, materials, methods and techniques described herein may, in some embodiments, be similar to any one or more of the embodiments, features, systems, devices, materials, methods and techniques described in the following:
U.S. application Ser. No. 16/048,167, filed on Jul. 27, 2017, titled “SYSTEMS AND METHODS FOR ASSISTING AND AUGMENTING SURGICAL PROCEDURES;”
U.S. application Ser. No. 16/242,877, filed on Jan. 8, 2019, titled “SYSTEMS AND METHODS OF ASSISTING A SURGEON WITH SCREW PLACEMENT DURING SPINAL SURGERY;”
U.S. application Ser. No. 16/207,116 (now U.S. Pat. No. 11,083,586), filed on Dec. 1, 2018, titled “SYSTEMS AND METHODS FOR MULTI-PLANAR ORTHOPEDIC ALIGNMENT;”
U.S. application Ser. No. 16/352,699, filed on Mar. 13, 2019, titled “SYSTEMS AND METHODS FOR ORTHOPEDIC IMPLANT FIXATION;”
U.S. application Ser. No. 16/383,215, filed on Apr. 12, 2019, titled “SYSTEMS AND METHODS FOR ORTHOPEDIC IMPLANT FIXATION;”
U.S. application Ser. No. 16/569,494, filed on Sep. 12, 2019, titled “SYSTEMS AND METHODS FOR ORTHOPEDIC IMPLANTS;”
U.S. Application No. 62/773,127, filed on Nov. 29, 2018, titled “SYSTEMS AND METHODS FOR ORTHOPEDIC IMPLANTS;”
U.S. application Ser. No. 16/699,447, filed on Nov. 29, 2019, titled “SYSTEMS AND METHODS FOR ORTHOPEDIC IMPLANTS”
U.S. Application No. 62/928,909, filed on Oct. 31, 2019, titled “SYSTEMS AND METHODS FOR DESIGNING ORTHOPEDIC IMPLANTS BASED ON TISSUE CHARACTERISTICS;”
U.S. application Ser. No. 16/735,222 (now U.S. Pat. No. 10,902,944), filed Jan. 6, 2020, titled “PATIENT-SPECIFIC MEDICAL PROCEDURES AND DEVICES, AND ASSOCIATED SYSTEMS AND METHODS;”
U.S. application Ser. No. 17/342,439 (now U.S. Pat. No. 11,376,076), filed Jun. 8, 2021, titled “PATIENT-SPECIFIC MEDICAL PROCEDURES AND DEVICES, AND ASSOCIATED SYSTEMS AND METHODS;”
U.S. application Ser. No. 16/987,113, filed Aug. 6, 2020, titled “PATIENT-SPECIFIC ARTIFICIAL DISCS, IMPLANTS AND ASSOCIATED SYSTEMS AND METHODS;”
U.S. application Ser. No. 16/990,810, filed Aug. 11, 2020, titled “LINKING PATIENT-SPECIFIC MEDICAL DEVICES WITH PATIENT-SPECIFIC DATA, AND ASSOCIATED SYSTEMS, DEVICES, AND METHODS;”
U.S. application Ser. No. 17/085,564, filed Oct. 30, 2020, titled “SYSTEMS AND METHODS FOR DESIGNING ORTHOPEDIC IMPLANTS BASED ON TISSUE CHARACTERISTICS;”
U.S. application Ser. No. 17/100,396, filed Nov. 20, 2020, titled “PATIENT-SPECIFIC VERTEBRAL IMPLANTS WITH POSITIONING FEATURES;”
U.S. Application No. 63/223,827, filed Jul. 20, 2021, titled “SYSTEMS FOR PREDICTING INTRAOPERATIVE PATIENT MOBILITY AND IDENTIFYING MOBILITY-RELATED SURGICAL STEPS;”
U.S. application Ser. No. 17/124,822, filed Dec. 17, 2020, titled “PATIENT-SPECIFIC MEDICAL PROCEDURES AND DEVICES, AND ASSOCIATED SYSTEMS AND METHODS;”
U.S. Application No. 63/274,300, filed Nov. 1, 2022, titled “PATIENT-SPECIFIC SPINAL INSTRUMENTS AND DECOMPRESSION SYSTEMS;” and
International Application No. PCT/US2021/012065, filed Jan. 4, 2021, titled “PATIENT-SPECIFIC MEDICAL PROCEDURES AND DEVICES, AND ASSOCIATED SYSTEMS AND METHODS.”

All of the above-identified patents and applications are incorporated by reference in their entireties. In addition, the embodiments, features, systems, devices, materials, methods and techniques described herein may, in certain embodiments, be applied to or used in connection with any one or more of the embodiments, features, systems, devices, or other matter.

The ranges disclosed herein also encompass any and all overlap, sub-ranges, and combinations thereof. Language such as “up to,” “at least,” “greater than,” “less than,” “between,” or the like includes the number recited. Numbers preceded by a term such as “approximately,” “about,” and “substantially” as used herein include the recited numbers (e.g., about 10%=10%), and also represent an amount close to the stated amount that still performs a desired function or achieves a desired result. For example, the terms “approximately,” “about,” and “substantially” may refer to an amount that is within less than 10% of, within less than 5% of, within less than 1% of, within less than 0.1% of, and within less than 0.01% of the stated amount.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting.

What is claimed is:

1. A computer-implemented method comprising:
receiving patient data for a patient, the patient data including one or more images of the patient's spinal region showing the patient's anatomy;
identifying at least one site of nerve compression and target tissue at least partially contributing to the nerve compression based on the received patient data; and
designing at least one patient-specific instrument configured to access the target tissue and perform a decompression step on the target tissue.

2. The computer-implemented method of claim 1, further comprising:
determining an access path for surgically accessing the target tissue, wherein the at least one patient-specific instrument is designed to access the target tissue using the access path;
generating a personalized surgical plan for implanting one or more patient-specific interbody devices for achieving a target corrected spinal configuration for a segment of the patient's spine; and
generating a decompression plan for utilizing the at least one patient-specific instrument in conjunction with implantation of the one or more patient-specific interbody devices.

3. The computer-implemented method of claim 1, further comprising:
generating a three-dimensional virtual model representing the patient's spinal region; and
designing, using the three-dimensional virtual model, at least one surgical site preparation instrument configured to prepare an implantation site for an implant.

4. The computer-implemented method of claim 3, further comprising designing the at least one patient-specific instrument and the at least one surgical site preparation instrument to be passed through a cannula positioned in the patient or an incision in the patient.

5. The computer-implemented method of claim 3, further comprising:
designing the at least one patient-specific instrument to be delivered through a first cannula positioned in the patient; and
designing the at least one surgical site preparation instrument to be delivered through a second cannula positioned in the patient and spaced apart from the first cannula.

6. The computer-implemented method of claim 1, wherein designing the at least one patient-specific instrument includes:
determining an access path to the target tissue;
designing a distal head with an atraumatic region configured to face nerve tissue when the distal head removes the target tissue; and
designing an elongated body connected to the distal head and configured to extend along the access path to position the distal head at the at least one site and enable removal of the target tissue.

7. The computer-implemented method of claim 6, wherein the distal head is a debulking head, a rongeur head, a cutting head, or a reamer.

8. The computer-implemented method of claim 1, further comprising determining a safe working zone for performing the decompression step,
wherein designing the at least one patient-specific instrument includes configuring a distal end of the at least one patient-specific instrument to remain in the safe working zone when performing the decompression step.

9. The computer-implemented method of claim 1, wherein the patient data further includes pain data, the method comprising:
identifying one or more candidate nerve compression sites, and
correlating the pain data with the one or more candidate nerve compression sites to identify at least one of the candidate nerve compression sites as the at least one site of nerve compression.

10. The computer-implemented method of claim 1, wherein designing the at least one patient-specific instrument includes:
determining a sequence of steps to remove the target tissue; and
designing a plurality of patient-specific instruments each configured to perform at least one of the steps in the sequence.

11. The computer-implemented method of claim 1, further comprising:
generating a virtual model of the patient's spinal region;
simulating, using the virtual model, spinal motion to determine motion related spinal nerve compression based on different loading conditions; and
generating a surgical decompression plan including one or more images of the virtual model of the spinal region in which nerve tissue is decompressed,
one or more pain reduction scores for the surgical decompression plan, and/or
identification of one or more target levels for decompression.

12. The computer-implemented method of claim 11, wherein the surgical decompression plan identifies one or more safe working zones in which the at least one patient-specific instrument can be positioned to perform the decompression step.

13. The computer-implemented method of claim 1, further comprising generating a surgical decompression plan that identifies tissues, tissue margins, and/or nerve compression sites.

14. The computer-implemented method of claim 1, further comprising:
receiving user input for hand movement;
determining one or more manipulation parameters based on the user input; and
designing the at least one patient-specific instrument based on the determined one or more manipulation parameters.

15. The computer-implemented method of claim 14, further comprising:
simulating a surgical procedure using hand input devices operated by the user to
generate the user input, wherein the user input includes one or more of
at least one range of motion of one or both hands of the user,
the user's strength, and/or
the user's manual instrument positioning accuracy.

16. The computer-implemented method of claim 1, wherein the at least one patient-specific instrument includes a set of instruments each configured to remove tissue from the target site.

17. The computer-implemented method of claim 16, further comprising:
determining a sequence for using the instruments in the set based on at least one procedure threshold.

18. The computer-implemented method of claim 17, wherein the at least one procedure threshold includes a safety threshold, a time threshold, and/or predicted outcome threshold.

19. A system for a patient-specific spinal decompression procedure, the system comprising:
one or more processors; and
a memory storing instructions that, when executed by the one or more processors, cause the system to perform operations comprising:
receiving patient data for a patient, the patient data including one or more images of the patient's anatomy;
identifying at least one site of nerve compression and target tissued at least partially contributing to the nerve compression based on the received patient data;
designing at least one patient-specific instrument configured to access the target tissue and perform a decompression step on the target tissue.

20. The system of claim 19, wherein the operations further include
generating a personalized surgical plan for implanting one or more patient-specific interbody devices for achieving a target corrected spinal configuration for a segment of the patient's spine; and
generating a decompression plan for utilizing the at least one patient-specific instrument in conjunction with implantation of the one or more patient-specific interbody devices.

21. The system of claim 19, wherein the operations further include designing the at least one patient-specific instrument to include:
designing a distal head with an atraumatic region configured to face nerve tissue when the distal head removes the target tissue from the at least one site; and
designing an elongated body connected to the distal head and configured to extend along an access path to position the distal head at the at least one site.

22. The system of claim 21, wherein the distal head is a debulking head, a rongeur head, a cutting head, or a reamer.

23. The system of claim 19, wherein the operations further include determining a safe working zone for performing the decompression step,
wherein designing the at least one patient-specific instrument includes configuring a distal end of the at least one patient-specific instrument to remain in the safe working zone when performing the decompression step.

24. The system of claim 19, wherein the patient data further includes pain data, the system comprising:
identifying one or more candidate nerve compression sites, and
correlating the pain data with the one or more candidate nerve compression sites to identify at least one of the one or more candidate nerve compression sites as the at least one site of nerve compression.

25. The system of claim 19, wherein designing the at least one patient-specific instrument includes:
determining a sequence of steps to remove the target tissue; and
designing a plurality of patient-specific instruments each designed based on the patient's anatomy.

26. The system of claim 19, wherein the operations further include:
generating a virtual model of the spinal region to illustrate the spine;
simulating, via the virtual model, spinal motion to determine motion related spinal nerve compression based on different loading conditions; and
generating a surgical decompression plan including
one or more images of the virtual model of the spinal region in which nerve tissue is decompressed,
one or more predicted pain reduction scores for the surgical decompression plan, and/or
identification of one or more target levels for decompression.

27. The system of claim 19, wherein the operations further include:
receiving user input for hand movement;
determining one or more manipulation parameters based on the user input; and
designing the at least one patient-specific instrument based on the determined one or more manipulation parameters.

28. The system of claim 19, wherein the operations further include:
simulating a surgical procedure using hand input devices operated by the user to
generate the user input, wherein the user input includes one or more of
at least one range of motion of one or both hands of the user,
the user's strength, and/or
the user's manual instrument positioning accuracy.

29. The system of claim 19, wherein the at least one patient-specific instrument includes a set of instruments each configured to remove tissue from the target site.

30. The system of claim 29, wherein the operations further include:
determining a sequence for using the instruments in the set based on at least one procedure threshold.

31. The system of claim 30, wherein the at least one procedure threshold includes a safety threshold, a time threshold, and/or predicted outcome threshold.

32. A computer-readable storage medium storing instructions that, when executed by a computing system, cause the computing system to perform operations comprising:
receiving patient data for a patient, the patient data including one or more images of the patient's spinal region showing the patient's anatomy;
identifying at least one site of nerve compression and target tissued at least partially contributing to the nerve compression based on the received patient data;
determining an access path for surgically accessing the target tissue; and
designing at least one patient-specific instrument configured to access the target tissue via the access path and to perform a decompression step on the target tissue.

33. A computer-readable storage medium of claim 32, wherein the operations further include:
generating a personalized surgical plan for implanting one or more patient-specific interbody devices for achieving a target corrected spinal configuration for a segment of patient's spine; and
generating a decompression plan for utilizing the at least one patient-specific instruments in conjunction with implantation of the one or more patient-specific interbody devices.

34. A computer-implemented method comprising:
receiving patient data for a patient, the patient data including one or more images of anatomy of the patient;
determining a surgical plan with one or more spinal corrections for the patient's pre-operative spine for achieving a target corrected spinal configuration based on the patient data; and
predicting and compensating for post-operative nerve compression associated with the one or more spinal corrections.

35. The computer-implemented method of claim 34, wherein predicting and compensating for the post-operative nerve compression includes:
comparing a pre-operative configuration of the patient's vertebrae to a post-operative target configuration of the vertebrae;
predicting post-operative nerve compression that is caused by the one or more spinal corrections and exceeds a threshold level based on the comparison; and
generating a decompression plan to compensate for the predicted post-operative nerve compression.

36. The computer-implemented method of claim 34, further comprising:
predicting nerve compression; and
designing at least one patient-specific instrument configured to perform one or more decompression steps based on the predicted nerve compression.

37. The computer-implemented method of claim 34, further comprising:
generating a personalized surgical plan for implanting one or more patient-specific interbody devices for achieving the target corrected spinal configuration for a segment of patient's spine.

38. The computer-implemented method of claim 34, wherein predicting and compensating for the post-operative nerve compression includes:
simulating the one or more spinal corrections by moving features of a three-dimensional virtual model of the patient's anatomy;
identifying compression of nerve tissue caused by the movement of the features of the three-dimensional virtual model; and
modifying the surgical plan to reduce the identified compression of nerve tissue.

39. The computer-implemented method of claim 34, wherein predicting and compensating for the post-operative nerve compression includes:
virtually simulating nerve compression caused by the one or more spinal corrections; and
generating a decompression plan based on the virtual simulation of nerve tissue.

40. The computer-implemented method of claim 34, further comprising designing a set of patient-specific decompression instruments based on the surgical plan.

41. The computer-implemented method of claim 34, further comprising:
predicting intra-operative and/or post-operative nerve compression associated with the surgical plan; and
determining, based on the predicted intra-operative and/or the post-operative nerve compression, whether to modify the surgical plan, recommend a decompression procedure, or both.

42. The computer-implemented method of claim 34, further comprising in response to receiving user approval of a decompression procedure of the surgical plan, designing one or more patient-specific decompression instruments.

43. The computer-implemented method of claim 34, further comprising predicting and compensating for one or more additional post-operative adverse effects by at least one of modifying the surgical plan or generating additional surgical steps.

44. The computer-implemented method of claim 34, further comprising:
identifying pre-operative nerve compression causing pain and/or discomfort based on pain data of the patient; and
predicting that the nerve compression will remain post-operatively when the one or more spinal corrections do not cause significant reduction of the nerve compression.

45. The computer-implemented method of claim 34, further comprising collecting post-operative pain data for training a machine-learning model that was used to predict the post-operative nerve compression.

46. The computer-implemented method of claim 34, further comprising designing one or more patient-specific instruments based on a pre-operative spinal configuration of the patient, a predicted intra-operative spinal configuration of the patient, and/or the target corrected spinal configuration of the patient.

47. The computer-implemented method of claim 34, further comprising determining a level-by-level nerve compression score for the surgical plan for display to a user.

48. The computer-implemented method of claim 34, further comprising generating a decompression plan for substantially eliminated targeted nerve compression.

49. The computer-implemented method of claim 48, further comprising identifying targeted tissue that can be removed from the patient to achieve the elimination of the targeted nerve compression.

50. The computer-implemented method of claim 48, further comprising predicting a pain reduction score for a decompression procedure.

51. The computer-implemented method of claim 34, further comprising:
predicting a first pain reduction score for removing tissue associated with nerve compression according to the surgical plan, and
predicting a second pain reduction score for implantation of at least one implant according to the surgical plan.

52. The computer-implemented method of claim 34, further comprising:
designing a surgical kit for performing the surgical plan, wherein the surgical kit includes one or more instruments and one or more implants.

53. The computer-implemented method of claim 52, wherein
the one or more instruments are designed based on the one or more implants; and
the one or more implants are designed based on the one or more instruments.

54. The computer-implemented method of claim 52, further comprising:
sending instructions to manufacture the kit; and
validating that the manufactured kit is complete.
